(19) United States
(12) Patent Application Publication
(10) Pub. No.: US 2026/0265055 A1
(43) Pub. Date: Sep. 10, 2026

(54) Sulfine-Based Compounds for Generating Hydrogen Persulfide

(71) Applicant: Brown University, Providence, RI (US)

(72) Inventors: Ming Xian, Barrington, RI (US); Qi Cui, Ithaca, NY (US); Meg Shieh, Providence, RI (US)

(21) Appl. No.: 19/165,221

(22) PCT Filed: Mar. 15, 2024
(86) PCT No.: PCT/US2024/020078

§ 371 Date: Sep. 15, 2025

Related U.S. Application Data
(60) Provisional application No. 63/490,602, filed on Mar. 16, 2023.

(51) Int. Cl. C01B 17/18; C07D 335/06; C07D 339/04

(52) U.S. Cl. CPC C01B 17/18; C07D 335/06; C07D 339/04; C01P 2002/84; C01P 2002/86; C01P 2002/89

(57) ABSTRACT

Embodiments described herein are directed to compounds and methods for generating hydrogen persulfide. In particular, the disclosure provides compounds including a stable sulfine moiety which reacts with hydrogen sulfide, such as at a site within an individual, and ultimately generates hydrogen persulfide, such as in vivo within the individual.

### FEDERALLY SPONSORED RESEARCH AND DEVELOPMENT

This invention was made with government support under R01GM125968 from the National Institutes of Health. The government has certain rights in the invention.

### TECHNICAL FIELD

The present disclosure relates in general to sulfine-based compounds and their use to generate hydrogen persulfide.

### DESCRIPTION OF THE RELATED ART

Hydrogen persulfide (H₂S₂) is an important regulator in redox biology and is closely linked to H₂S-signaling. See Kimura, H.: Physiological roles of hydrogen sulfide and polysulfides. In Chemistry, Biochemistry and Pharmacology of Hydrogen Sulfide; Moore, P. K., Whiteman, M., Eds.; Springer International Publishing: Cham, 2015; pp 61-81; Liu, H. et al., Inorganic hydrogen polysulfides: chemistry, chemical biology, and detection. Br J. Pharmacol. 2019, 176, 616-627; Akaike, T. et al., Cysteinyl-tRNA synthetase governs cysteine polysulfidation and mitochondrial bioenergetics. Nat. Commun. 2017, 8, 1177. Endogenous H₂S₂ is produced by enzymes like 3-mercaptopyruvate transferase (3-MST) and cysteinyl-tRNA synthetases (CARS). It can also be produced from H₂S via H₂S—NO cross talk or hemeprotein-catalyzed oxidation. H₂S₂ appears to be more efficient than H₂S in inducing protein persulfidation (forming P—SSH), and this is suggested to be one of the main functions of H₂S₂ in biology. See Greiner, R. et al., Polysulfides link H₂S to protein thiol oxidation. Antioxid. Redox Signal. 2013, 19, 1749-1765; Filipovic, M. R. et al., Chemical biology of H₂S signaling through persulfidation. Chem. Rev. 2018, 118, 1253-1337; Yang, C. T. et al., S-Persulfidation: chemistry, chemical biology, and significance in health and disease. Antioxid. Redox Signal. 2020, 33, 1092-1114; Ida, T. et al., Reactive cysteine persulfides and S-polythiolation regulate oxidative stress and redox signaling. Proc. Natl. Acad. Sci. USA 2014, 111, 7606-7611.

However, the study of H₂S₂ in biological settings is still challenging due to its instability and high reactivity, as well as the lack of suitable donor compounds available to produce H₂S₂. Currently, most studies use Na₂S₂ as the equivalent of H₂S₂, but Na₂S₂ is considered an uncontrollable and instant H₂S₂ donor. Synthetic H₂S₂ donors have been reported. Xu, S. et al., Strategies for the design of donors and precursors of reactive sulfur species. Chem. Eur J. 2019, 25, 4005-4016; Xu, S. et al., Diacyl disulfides as the precursors for hydrogen persulfides (H₂S₂). Bioorg. Med. Chem. Lett. 2020, 30, 126903; Kang, J. et al., O→S relay deprotection, a general approach for the design of controllable donors of reactive sulfur species. Angew. Chem. Int. Ed. 2018, 57, 5893-5897; Yu, B. et al., Toward direct protein S-persulfidation: a prodrug approach that directly delivers hydrogen persulfide. J. Am. Chem. Soc. 2018, 140, 30-33. Exemplary structures are shown below.

Reported H₂S₂ Donors:

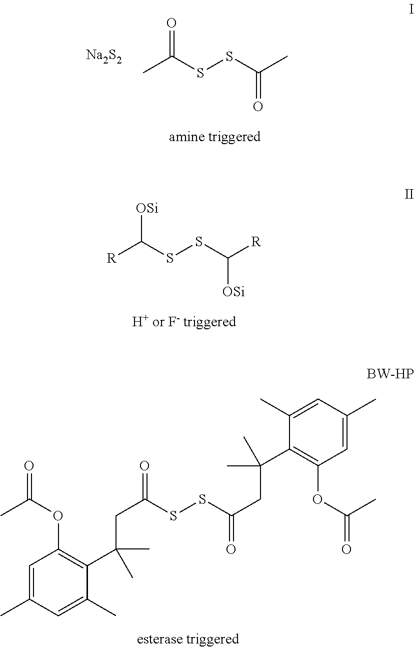

These donors rely on protected disulfide (—S—S—) structures, which have inherent limitations. For example, disulfides are sensitive to disulfide-exchange reactions with cellular thiols (cysteine or glutathione (GSH)). Such reactions would change their identity (e.g. not form H₂S₂). Acyl disulfides (compound I and compound BW-HP shown above) are highly reactive toward cellular nucleophiles (—SH, —NH₂, etc.), which can diminish their efficiency.

In theory, H₂S₂ can be produced from in situ reactions with oxidants such as H₂O₂ and HClO₄. See Li, Q. et al., Chemical foundation of hydrogen sulfide biology. Nitric Oxide, 2013, 35, 21-34. However, such H₂S₂ delivery for research or therapeutic purposes is not advantageous as the presence of oxidants could compromise the biological systems under investigation or being treated. In addition, the oxidation is difficult to control, so over-oxidation could occur to produce the stable and much less reactive adduct-elemental sulfur (S₈). The specificity of oxidation is also a problem as many biomolecules (such as proteins) could be modified by oxidation conditions.

Accordingly, compounds for the production of H₂S₂ which are more beneficial than current methods of H₂S₂ production are needed for research and therapeutic use.

### SUMMARY OF THE INVENTION

Aspects of the present disclosure are directed to compounds and methods for generating hydrogen persulfide. According to one aspect, compounds and methods are described for converting hydrogen sulfide to hydrogen persulfide. Compounds and methods for generating or delivering hydrogen persulfide as described herein may be for research purposes or used as a research tool, therapeutic purposes as a therapeutic treatment option, or any other industrial or manufacturing purpose where the generation of hydrogen persulfide is advantageous. According to the present disclosure, the H₂S₂ may be generated in vitro or in vivo.

According to one aspect, the present disclosure provides sulfine compounds which participate in a reaction to convert H₂S to H₂S₂ via non-oxidation and non-disulfide conditions, within a reaction medium. Exemplary compounds can be used to produce H₂S₂ for researching biological systems by participating in a reaction to generate H₂S₂ within a biological system of interest whether in vitro or in vivo. Exemplary compounds can be used to produce H₂S₂ for therapeutic applications by participating in a reaction to generate H₂S₂ at a therapeutically beneficial site within an individual in need of such treatment. Exemplary compounds can be administered to a reaction site of interest as a pharmaceutical composition or within a suitable delivery vehicle or excipient. Exemplary compounds can be used to produce H₂S₂ for any purpose where the generation of H₂S₂ is beneficial.

According to one aspect, H₂S used in the reaction with exemplary compounds to generate H₂S₂ can be native to a reaction site or provided/administered directly or via an H₂S donor compound. H₂S donor compounds are known to those of skill in the art or may be identified by literature search. In this aspect, the H₂S donor compound generates H₂S which is then reacted with exemplary compounds as described herein. According to one embodiment, the H₂S donor compound may be conjugated, linked, covalently bound or otherwise attached to an exemplary compound as described herein. In this manner, the H₂S generated by the H₂S donor compound is localized at or near the exemplary compound thereby facilitating reaction.

According to one aspect, exemplary compounds of the present disclosure can be conjugated, linked, covalently bound or otherwise attached to a protein, ligand, antibody or other delivery molecule, device or particle. For example, an exemplary compound can be bound to one protein of a protein-protein binding pair. In this manner, the exemplary compound can be targeted to a location where the other protein of the protein-protein binding pair is present. Protein-protein binding pairs are known to those of skill in the art. For example, an exemplary compound can be bound to a receptor/ligand of a receptor/ligand-ligand binding pair. In this manner, the exemplary compound can be targeted to a location where the other ligand of the receptor/ligand-ligand binding pair is present. Receptor/Ligand-ligand binding pairs are known to those of skill in the art. For example, an exemplary compound can be bound to an antibody to an antigen. In this manner, the exemplary compound can be targeted to a location where the antigen is present. Antibody-antigen binding pairs are known to those of skill in the art. For example, the exemplary compound can be bound to a delivery molecule, delivery device or delivery particle for the delivery of the exemplary compound to a desired site, such as a location within a mammal, such as a human. Delivery molecules, delivery devices or delivery particles for delivery of active agents are known to those of skill in the art, and can be used to deliver the exemplary compounds.

According to one aspect, exemplary compounds of the present disclosure can be used in the generation of H₂S₂ and can then be regenerated by a reactive oxygen species after being used to produce H₂S₂. The reactive oxygen species can be native to the reaction site, such as naturally present within an individual, or can be introduced, administered or delivered as a reagent to the reaction site, whether as a native or non-native species.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1A depicts UV-Vis absorbance spectra changes of the reaction between TTS (100 μM) and H₂S (200 μM) over 1 min intervals.

FIG. 1B depicts representative time-dependent [TTS] changes based on UV-Vis absorption changes at 380 nm of the reaction between TTS (50 μM) and Na₂S (5 mM).

FIG. 1C depicts representative time-dependent −ln([TTS]/[TTS]₀) changes based on UV-Vis absorption changes at 380 nm of the reaction between TTS (50 μM) and Na₂S (5 mM).

FIG. 2A depicts data of fluorescence enhancements of SSP4 (5 μM) in the presence of: (1) SSP4 only; (2) 25 μM TTS; (3) 50 μM Na₂S; (4) 100 μM Cys; (5) 25 μM TT; (6) 25 μM TTS+50 μM Na₂S; (7) 25 μM TTS+100 μM Cys; (8) 25 μM TT+50 μM Na₂S; (9) 25 μM TT+100 μM Cys; (10) 25 μM Na₂S₂. Results are expressed as mean±SEM (n=3).

FIG. 2B depicts data of percent fluorescence of (11) 25 μM TTS+200 μM Cys; (12) 25 μM TTS+1 mM GSH; (13) 25 μM TTS+50 μM Na₂S+200 μM Cys; (14) 25 μM TTS+50 μM Na₂S+1 mM GSH; (15) 25 μM TT+200 μM Cys; (16) 25 μM TT+1 mM GSH; (17) 25 μM TT+50 μM Na₂S+200 μM Cys; (18) 25 μM TT+50 μM Na₂S+1 mM GSH compared to (6). Results are expressed as mean±SEM (n=3).

FIG. 3 depicts NMR spectra changes of the oxidation of TT to form TTS.

FIGS. 4A-G depict representative fluorescence images of the reaction between TTS and H₂S in HeLa cells. Cells were first treated with (FIG. 4A) TTS only (50 μM), (FIG. 4B) TTS (50 μM) and Na₂S (100 μM), (FIG. 4C) TT only (50 μM), (FIG. 4D) Na₂S₂ only (50 μM), (FIG. 4E) TT (50 μM) and Na₂S (100 μM), (FIG. 4F) Na₂S only (100 μM) or (FIG. 4G) no treatment for 30 min and washed twice with PBS. Cells were then treated with SSP4 (5 μM) and CTAB (100 μM) for 30 min and washed three times with PBS before being subjected to imaging. Top image is green channel imaging. Bottom image is brightfield imaging. Scale bars, 100 m. FIG. 4H is a graph of fluorescence intensities of SSP4 staining. Data are mean values ±SEM calculated using ImageJ. (n=9, where three different images were obtained from each of three samples and quantified).

FIG. 5 depicts TTS-conjugated lysozyme reactions and MS spectra.

FIG. 6A depicts data for the fluorescence enhancement of SSP4 for the detection of persulfidated GAPDH when the reduced protein (100 μM) was treated with: (1) control (no treatment); (2) Na₂S (1 mM); (3) TTS (1 mM); (4) TTS (1 mM)+Na₂S (1 mM); (5) Na₂S₂ (1 mM). Results are expressed as mean±SEM (n=3). Statistical analysis was performed using one-way ANOVA. ****P<0.0001.

FIG. 6B depicts data of the fluorescence enhancement of SSP4 (5 μM) for GAPDH (10 μM, after Zeba column desalting and dilution) when the reduced protein (100 μM) was treated with: (1) control (no treatment); NEM followed by (2) TTS (1 mM)+Na₂S (1 mM) or (3) Na₂S₂ (1 mM); or small molecules were added to Zeba columns without protein (4) TTS (1 mM)+Na₂S (1 mM); (5) Na₂S₂ (1 mM). Results are expressed as mean±SEM (n=3).

FIG. 6C depicts the tandem mass spectra (MS2): Spectrum of a GAPDH peptide GILGYTEDQVVSC(282)@DFNSDTHSSTFDAGAGIALNDHFVK persulfidated at Cys282 with the TTS+Na₂S system. Fragment ions containing persulfidation include y26++, y27++, y28++, and y29++.

FIG. 6D depicts the tandem mass spectra (MS2): Spectrum of a GAPDH peptide IVSNASC(150)@TTNC(154)@LAPLAK persulfidated at both Cys150 and Cys154 with the TTS+Na₂S system. Fragment ions containing persulfidation include y11+, y12+, y13+, and y15+. The symbol @ indicates persulfidation.

FIG. 7 depicts data for cell viability versus concentration of TTS and TT in HeLa cells with the CCK-8 assay. Results are expressed as mean±SEM (n=3).

FIG. 8 depicts data of fluorescence response of SSP4 for the reaction between Lyso-TTS and Na₂S. a: Lyso-TTS/Na₂S reaction solution 50 μL, PBS buffer (pH 7.4, 47 μL), SSP4 (1 mM in DMSO, 1 μL), CTAB (5 mM in EtOH, 2 μL). b: Lyso-TT solution (80 μM, 45 μL), PBS buffer (pH 7.4, 52 μL), SSP4 (1 mM in DMSO, 1 μL), CTAB (5 mM in EtOH, 2 μL).

FIG. 9A depicts reactions of H₂S donor compound JK-1 to produce H₂S and the use of JK-1 as an H₂S donor compound with TTS to generate H₂S₂.

FIG. 9B depicts data of fluorescence enhancements of SSP4 (40 μM) in the presence of: (1) SSP4 only; (2) TTS (200 μM); (3) JK-1 (400 μM); (4) TTS (200 μM)+JK-1 (400 μM); (5) Na₂S₂ (200 μM). Results are expressed as mean±SEM (n=3).

FIG. 10 depicts reactions of H₂S donor compound TAGDD-6 to produce H₂S in the presence of cysteine and the use of a conjugate of TAGDD-6 and TTS to generate H₂S₂.

FIG. 11A depicts data of time dependent fluorescence response over 12 hours.

FIG. 11B depicts data of fluorescence enhancement at the 12 hour time point of 1) SSP4 only (5 μM); 2) 3 only (25 μM); 3) Cys only (100 μM); 4) TTS only (25 μM); 5) 3 (25 μM)+Cys (100 μM); 6) Na₂S₂ (25 μM). Compounds were first incubated in 50 mM PBS (pH 7.4) for 30 minutes in the dark at room temperature (“rt”) before SSP4 (5 μM) and CTAB (50 μM) were added and incubated for another 30 min. Results are expressed as mean±SEM (n=3).

### DETAILED DESCRIPTION

The present disclosure provides compounds and methods for generating hydrogen persulfide (H₂S₂). According to one aspect, the compounds and methods of the present disclosure generate hydrogen persulfide from hydrogen sulfide. The hydrogen sulfide may be provided by a hydrogen sulfide donor compound.

According to one aspect, the compounds of the present disclosure include a sulfine (thiocarbonyl oxide) moiety. According to one aspect, the compounds of the present disclosure comprise a stable sulfine (thiocarbonyl oxide) moiety. Stability is understood as referring to the half life of the sulfine moiety. A longer half life indicates greater stability.

According to one aspect, a compound comprises an aromatic heterocycle including a sulfine moiety. According to one aspect the stability or half life of the sulfine moiety is increased when present as part of an aromatic heterocycle relative to, for example, an aliphatic sulfine. According to one aspect, the sulfine moiety is stabilized (i.e., has a longer half life) insofar as being included with or in an aromatic heterocycle results in the sulfine moiety being relatively less reactive to cellular nucleophiles, such as —OH, —NH₂, etc., allowing more specific reactivity with hydrogen sulfide to generate hydrogen persulfide.

According to the present disclosure, aliphatic sulfines are normally unstable and decompose to form the corresponding carbonyl compounds and thiocarbonyl compounds with half-lives of 1-4 days. When UV light is introduced, atomic sulfur could be release. See Cerreta, F. et al., Carbon versus sulfur addition of nucleophilics to sulfines: the case of amines. Tetrahedron Lett. 1993, 34, 6741-6742; Chevrie, D. et al., Oxidation of dithiocarbamates and synthesis of a stable sulfine. Tetrahedron Lett. 1998, 39, 8983-8986; Akihiko Ishii, A. et al., Synthesis and photophysical properties of conjugated thioketone, thioketone S-oxide (Sulfine), and related compounds incorporated in a dibenzobarrelene skeleton. J. Sulfur Chem. 2020, 41, 238-257; Tamagaki, S. et al., Sulfines in the oxidation of 1,2-dithiole-3-thiones. Chem. Lett, 1980, 619-620. Nucleophiles like amines could react with sulfines to form imines and sulfenic acid (HSOH). Although sulfines may serve as sulfane sulfur donors, the poor stability of aliphatic sulfines limits their applications.

Aspects of the present disclosure are directed to increasing the stability or half life of a sulfine moiety, i.e. decreasing reactivity with cellular nucleophiles, wherein the carbon of the sulfine moiety (C═S═O) is part of an aromatic heterocycle. According to one aspect, a sulfine moiety is stabilized when part of an aromatic heterocyclic system such that the sulfine moiety is less reactive with cellular nucleophiles (—OH, —NH₂, etc.) thereby allowing a longer half life and greater ability to react with H₂S which may be present at or near a reaction site.

According to one aspect, compounds of the present disclosure including a sulfine moiety react with hydrogen sulfide to produce hydrogen persulfide (H₂S₂) (referred to as a “booster system” herein), which is useful for both research and therapeutic use or any manufacturing or industrial use where the generation of hydrogen persulfide is desired.

Regarding research or therapeutic use, as described in Cuevasanta et al., “Possible Molecular Basis of the Biochemical Effects of Cystein-derived Persulfides,” Frontiers in Molecular Biosciences, Vol. 9-2022; world wide website doi.org/10.3389/fmolb.2022.975988, persulfides (RSSH/RSS⁻) are species closely related to thiols (RSH/RS⁻) and hydrogen sulfide (H₂S/HS⁻), and can be formed in biological systems in both low and high molecular weight cysteine-containing compounds. They are key intermediates in catabolic and biosynthetic processes, and have been proposed to participate in the transduction of hydrogen sulfide effects.

They can be formed in biological systems through processes that are dependent or independent of H₂S/HS⁻, and they have prominent roles in sulfur trafficking and metabolism. They are intermediates in the mitochondrial oxidation of H₂S/HS⁻ and in the biosynthesis of thionucleosides, iron-sulfur clusters and other cofactors. Persulfides may also be possible intermediates in H₂S/HS⁻ signaling. Persulfides may also be involved with persulfidation (forming P—SSH) of proteins.

Accordingly, compounds which can be used to generate hydrogen persulfide are useful reagents in the study of systems utilizing hydrogen persulfide, are useful for therapeutic use where generation of hydrogen persulfide at a site within an individual can have a therapeutic effect, or for any other utility where generation of hydrogen persulfide is desirable.

### I. Exemplary Compounds

Compounds of the present disclosure include cyclic compounds, such as aromatic compounds, including a sulfine moiety as part of the cyclic compound, i.e. the carbon of the sulfine moiety is included in the ring structure of the cyclic compound. Compounds of the present disclosure include five membered or six membered cyclic compounds, such as aromatic compounds, including a sulfine moiety as part of the cyclic compound, i.e. the carbon of the sulfine moiety is included in the ring structure of the cyclic compound. The cyclic compound can be a heterocyclic compound, where a carbon atom may be substituted with an oxygen atom, a sulfur atom or a nitrogen atom. One or more carbon atoms within the cyclic compound may be substituted with an R group or one or more carbon atoms within the cyclic compound may be unsubstituted, i.e. hydrogen atoms are present as opposed to an R group.

Compounds of the present disclosure are generally described with reference to formula 1 below.

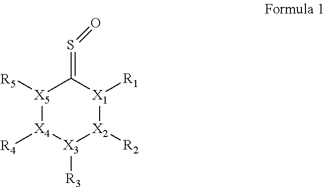

Formula 1 depicts a six-membered ring which may be aromatic or nonaromatic, substituted or unsubstituted, having 0, 1 or 2 two double bonds, wherein
X₁ is an oxygen atom, a nitrogen atom, a sulfur atom or a carbon atom, wherein R₁ is absent when X₁ is oxygen, sulfur or double bonded nitrogen;
X₂ is an oxygen atom, a nitrogen atom, a sulfur atom or a carbon atom, wherein R₂ is absent when X₂ is oxygen, sulfur or double bonded nitrogen;
X₃ is an oxygen atom, a nitrogen atom, a sulfur atom or a carbon atom, wherein R₃ is absent when X₃ is oxygen, sulfur or double bonded nitrogen;
X₄ is an oxygen atom, a nitrogen atom, a sulfur atom or a carbon, atom wherein R₄ is absent when X₄ is oxygen, sulfur or double bonded nitrogen;
X₅ is an oxygen atom, a nitrogen atom, a sulfur atom or a carbon atom, wherein R₅ is absent when X₅ is oxygen, sulfur or double bonded nitrogen;
with the proviso that no more than two of X₁, X₂, X₃, X₄, X₅ may be an atom other than carbon, i.e. a heteroatom,
R₁ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker;
R₂ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker;
R₃ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker;
R₄ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker; and
R₅ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker.
R₁, R₂, R₃, R₄, and R₅ may be the same or different. The R group or the linker may be bound to a protein, antibody, receptor, ligand, antibody, delivery molecule, device, particle or H₂S donating compound.

According to one aspect, compounds of the present disclosure are generally described with reference to formula 1A below.

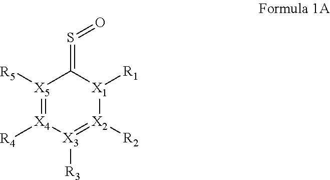

X₁ is an oxygen atom, a nitrogen atom, a sulfur atom or a carbon atom, wherein R₁ is absent when X₁ is oxygen, sulfur or double bonded nitrogen;
X₂ is an oxygen atom, a nitrogen atom, a sulfur atom or a carbon atom, wherein R₂ is absent when X₂ is oxygen, sulfur or double bonded nitrogen;
X₃ is an oxygen atom, a nitrogen atom, a sulfur atom or a carbon atom, wherein R₃ is absent when X₃ is oxygen, sulfur or double bonded nitrogen;
X₄ is an oxygen atom, a nitrogen atom, a sulfur atom or a carbon, atom wherein R₄ is absent when X₄ is oxygen, sulfur or double bonded nitrogen;
X₅ is an oxygen atom, a nitrogen atom, a sulfur atom or a carbon atom, wherein R₅ is absent when X₅ is oxygen, sulfur or double bonded nitrogen;
with the proviso that no more than two of X₁, X₂, X₃, X₄, X₅ may be an atom other than carbon, i.e. a heteroatom,
R₁ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker;
R₂ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker;
R₃ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker;
R₄ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker; and
R₅ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker.
R₁, R₂, R₃, R₄, and R₅ may be the same or different. The R group or the linker may be bound to a protein, antibody, receptor, ligand, antibody, delivery molecule, device, particle or H₂S donating compound.

An exemplary compound within Formula 1 and Formula 1A is

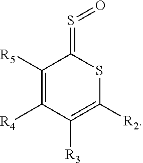

An exemplary compound within Formula 1 and Formula 1A is

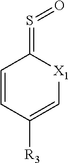

where X₁ is sulfur, oxygen or nitrogen.

An exemplary compound within Formula 1 and Formula 1A is

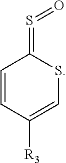

An exemplary compound within Formula 1 and Formula 1A is

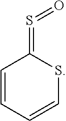

An exemplary compound within Formula 1 and Formula 1A is

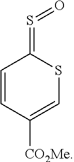 [SMILES: COC(=O)c1ccc(=S=O)sc1]

According to one aspect, R₅ and R₄ can be part of a fused ring structure such as a C5-C7 cyclic structure, saturated or unsaturated, heterocyclic or nonheterocyclic, substituted or unsubstituted. An exemplary fused ring structure is benzene or a C6 heterocycle including an oxygen atom, sulfur atom or nitrogen atom.

According to one aspect, R₄ and R₃ can be part of a fused ring structure such as a C5-C7 cyclic structure, saturated or unsaturated, heterocyclic or nonheterocyclic, substituted or unsubstituted. An exemplary fused ring structure is benzene or a C6 heterocycle including an oxygen atom, sulfur atom or nitrogen atom.

According to one aspect, R₃ and R₂ can be part of a fused ring structure such as a C5-C7 cyclic structure, saturated or unsaturated, heterocyclic or nonheterocyclic, substituted or unsubstituted. An exemplary fused ring structure is benzene or a C6 heterocycle including an oxygen atom, sulfur atom or nitrogen atom.

An exemplary compound with a fused ring structure is

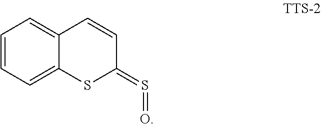 [SMILES: O=S=c1ccc2ccccc2s1]

As can be seen, exemplary compounds of Formula 1 and Formula 1A include a sulfine moiety, C═S═O, where the carbon of the sulfine moiety is within a six-membered ring such as a heterocycle, such as an aromatic heterocycle. According to one aspect, the sulfine moiety in compounds of the present disclosure is more stable, i.e. has a longer half life, insofar as it is less reactive to cellular nucleophiles (—OH, —NH₂, etc.) thereby allowing greater reactivity with H₂S.

### Representative Compounds Include

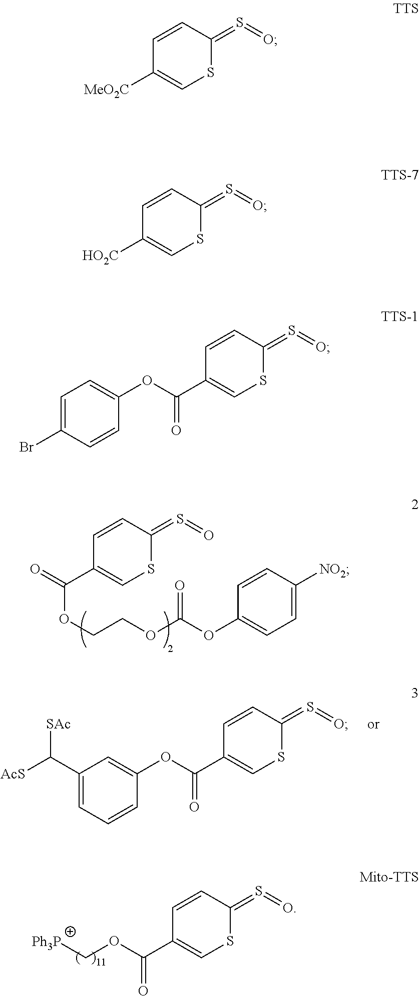

According to one aspect, compounds of the present disclosure are generally described with reference to formula 1B below where the sulfine moiety, C═S═O, is within a five-membered ring such as a heterocycle, such as an aromatic heterocycle.

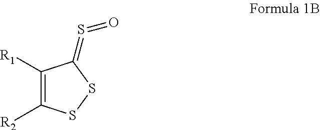

R₁ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker.

R₂ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker.

According to one aspect, R₁ and R₂ can be part of a fused ring structure such as a C5-C7 cyclic structure, saturated or unsaturated, heterocyclic or nonheterocyclic, substituted or unsubstituted. An exemplary fused ring structure is benzene or a C6 heterocycle including an oxygen atom, sulfur atom or nitrogen atom.

### Representative Compounds Include

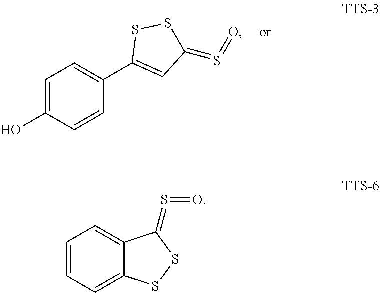 [SMILES: O=S=c1cc(-c2ccc(O)cc2)ss1 ; O=S=c1ssc2ccccc12]

Exemplary compounds as described herein which participate in a reaction to generate H₂S₂ may include a pharmaceutically acceptable salt thereof.

The term “alkyl” refers to straight or branched chain unsubstituted hydrocarbon groups of 1 to 20 carbon atoms, preferably 1 to 7 carbon atoms.

The term “substituted alkyl” refers to an alkyl group substituted by, for example, one to four substituents, such as, halo, alkanoyl, aryloxy, alkanoyloxy, amino, alkylamino, substituted alkylamino, cycloalkylamino, substituted cycloalkylamino, arylamino, substituted arylamino, aralkylamino, or substituted aralkyamino.

The term “aryl” refers to monocyclic or bicyclic aromatic hydrocarbon groups having 4 to 7 carbon atoms in the ring portion, such as phenyl, with each of which may be substituted.

The term “substituted aryl” refers to an aryl group substituted by, for example, one to four substituents such as alkyl, substituted alkyl, alkenyl, substituted alkenyl, alkynyl, substituted alkynyl, aryl, substituted aryl, aralkyl, halo, trifluoromethoxy, trifluoromethyl, alkanoyl, alkanoyloxy, aryloxy, aralkyloxy, amino, alkylamino, arylamino, aralkylamino, dialkylamino, alkanoylamino, thiol, alkylthio, ureido, nitro, cyano, carboxy, carboxyalkyl, carbamyl, alkoxycarbonyl, alkylthiono, arylthiono, arylsulfonylamine, sulfonic acid, alkysulfonyl, sulfonamido, aryloxy and the like.

The term “heteroaryl” refers to an optionally substituted, aromatic group for example, which is a 4 to 7 membered monocyclic, 7 to 11 membered bicyclic, or 10 to 15 membered tricyclic ring system, which has at least one heteroatom and at least one carbon atom-containing ring, for example, pyridine, tetrazole, or indazole.

The term “alkenyl” refers to straight or branched chain hydrocarbon groups of 2 to 20 carbon atoms, preferably 2 to 15 carbon atoms, and most preferably 2 to 8 carbon atoms.

The term “substituted alkenyl” refers to an alkenyl group substituted by, for example, one to two substituents, such as, halo, hydroxy, alkoxy, alkanoyl, alkanoyloxy, amino, alkylamino, dialkylamino, alkanoylamino, thiol, alkylthio, alkylthiono, alkylsulfonyl, sulfonamido, nitro, cyano, carboxy, carbamyl, substituted carbamyl, guanidino, indolyl, imidazolyl, furyl, thienyl, thiazolyl, pyrrolidyl, pyridyl, pyrimidyl and the like.

The term “alkynyl” refers to straight or branched chain hydrocarbon groups from 2 to 20 carbon atoms, and most preferably 2 to 8 carbon atoms.

The term “substituted alkynyl” refers to an alkynyl group substituted by a substituent, examples include; halo, hydroxy, alkoxy, alkanoyl, alkanoyloxy, amino, alkylamino, dialkylamino, alkanoylamino, thiol, alkylthio, alkylthiono, alkylsulfonyl, sulfonamido, nitro, cyano, carboxy, carbamyl, substituted carbamyl, guanidino and heterocyclyl, e.g. imidazolyl, furyl, thienyl, thiazolyl, pyrrolidyl, pyridyl, pyrimidyl and the like.

The term “cycloalkyl” refers to an optionally substituted, saturated cyclic hydrocarbon ring systems, preferably containing 1 to 3 rings and 3 to 7 carbons per ring which may be further fused with an unsaturated C3 C7 carbocyclic ring. Exemplary substituents include one or more alkyl groups as described above, or one or more groups described above as alkyl substituents.

The term “aralkyl” refers to an aryl or a substituted aryl group bonded directly through an alkyl group, such as benzyl or the like.

The terms “heterocycle”, “heterocyclic” and “heterocyclyl” refer to an optionally substituted, fully saturated or unsaturated, aromatic or nonaromatic cyclic group, which are a 4 to 7 membered monocyclic, which has at least one heteroatom in at least one carbon atom-containing ring. Each ring of the heterocyclic group containing a heteroatom may have at least one heteroatoms. The heterocyclic group may be attached at any heteroatom or carbon atom.

The term “heteroatoms” can include oxygen, sulfur and nitrogen.

The term “ring structure” refers to an organic cyclic compound in which an organic compound containing a series of atoms is connected to form a loop or ring. The term includes various cyclic compounds, which may be: saturated, unsaturated or aromatic; substituted or unsubstituted; hetero- or homo-cyclic; and may be mono- or polycyclic, as described herein. As used herein hetero-structures are those in which not all atoms of the primary structure are carbon. Instead, one or more are a different atom, for example a 6-membered ring in which 5 of the ring atoms are C and one is N.

According to one aspect, compounds of formula 1 convert hydrogen sulfide to hydrogen persulfide via non-oxidation and non-disulfide conditions.

Exemplary compounds of formula 1, such as 2H-thiopyran-2-thione sulfine (“TTS”) react with hydrogen sulfide and are converted into a thiocarbonyl-containing heterocycle, such as 2H-thiopyran-2-thione (TT). The thiocarbonyl-containing heterocycle, such as 2H-thiopyran-2-thione, can then react with a reactive oxygen species to produce the starting compound of formula 1. In this manner, compounds of formula 1 are regenerable. It is to be understood that “TTS” may refer to a substituted compound described herein or it may refer to the unsubstituted compound. “TTS” and “TT” are used for brevity and generally refer to a stabilized sulfine compound which can react with H₂S to generate H₂S₂ as described herein.

### III. Exemplary H₂S Donors

According to one aspect, a compound of Formula 1 or Formula 1A can react with H₂S which may be present at a reaction location. The H₂S may be generated or “donated” by a H₂S donor compound which can produce or donate H₂S. H₂S donor compounds are generally known in the art and generate H₂S, such as by hydrolysis, cellular thiol activation, or photolysis.

H₂S donating compounds are known to those of skill in the art as described in US 2017/253,622 (“Compounds for PH-controlled Release of Hydrogen Sulfide”) or U.S. Pat. No. 9,096,504 (“Controlled Chemical Release of Hydrogen Sulfide”) each of which is hereby incorporated by reference in its entirety for the teaching of H₂S donating compounds. Exemplary H₂S donating compounds described in US 2017/253,622 as releasing H₂S in a controlled system or biological systems including live cells include, but are not limited to, the following chemical structures below.

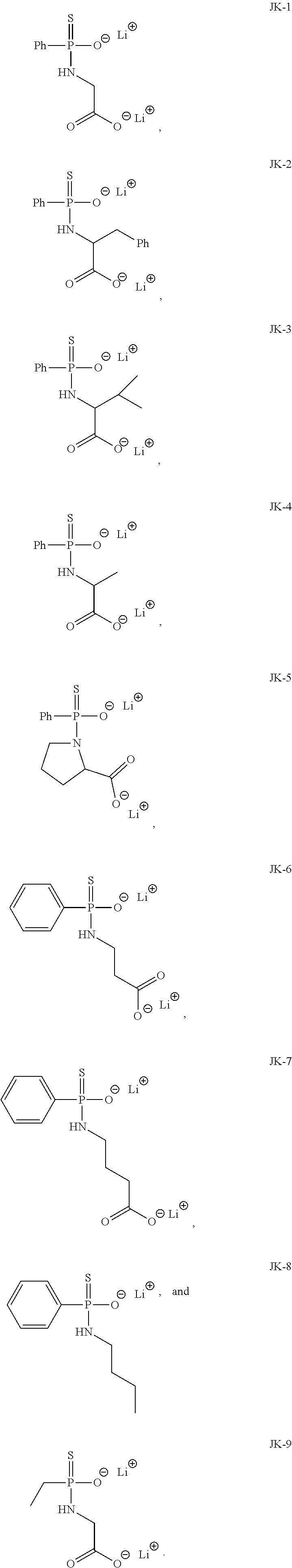

Additional representative H₂S donor compounds include those provided at Table 1 of U.S. Pat. No. 9,096,504 hereby incorporated by reference and having the general formula set forth below.

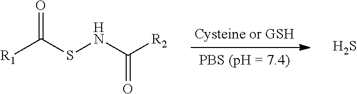

Additional representative species of H₂S donating compounds can be found in the following review articles: Zheng, Y. et al., Toward hydrogen sulfide based therapeutics: critical drug delivery and developability issues. Med. Res. Rev. 2018, 38, 57-100; Powell, C. R. et al., A review of hydrogen sulfide (H2S) donors: Chemistry and potential therapeutic applications. Biochem. Pharmacol. 2018, 149, 110-123; Zhao, Y. et al., Hydrogen sulfide (H2S) releasing agents: chemistry and biological applications. Chem. Commun. 2014, 50, 11788-11805, each of which are incorporated by reference herein for the teaching of hydrogen sulfide donating compounds useful in reacting with exemplary compounds described herein for the generation of hydrogen persulfide. In this manner, a compound of Formula 1 can react with H₂S generated, released or donated by an H₂S donating compound to result in production or generation of H₂S₂.

### IV. Exemplary Reactive Oxygen Species to Regenerate Sulfine-Containing Compounds

As described above, an exemplary compound of formula 1, such as 2H-thiopyran-2-thione sulfine (“TTS”) reacts with H₂S (native, provided/administered or generated from an H₂S donor) and is converted into a thiocarbonyl-containing heterocycle, such as 2H-thiopyran-2-thione (TT). The thiocarbonyl-containing heterocycle, such as 2H-thiopyran-2-thione, can then react with a reactive oxygen species (ROS) to produce the starting compound of Formula 1. In this manner, compounds of Formula 1 are regenerable. Exemplary reactive oxygen species are known to those of skill in the art or can be determined by literature search and include H₂02, O₂⁻, ^{t}BuOOH, AcOOH, SNP, OCl⁻, hydroxyl radical, etc. and the like.

### V. Sulfine-Containing Compounds Attached to a Delivery Molecule or Device

According to one aspect, a stabilized sulfine-containing compound, such as those of Formula 1, may be conjugated, linked, covalently bound or otherwise attached to a protein, receptor, ligand, antibody or other delivery molecule, device or particle. Linkers that can covalently react with a reactive amine group or a thiol group of the protein via common conjugation chemistry such as amidation, thiol-alkylation, imine formation, click chemistry, etc. and the like, as is known to those of skill in the art. Such linkers can also be covalently attached to a compound of Formula 1. In this aspect, the protein can serve to direct the compound of Formula 1 to a location of interest via a protein-protein interaction. The compound of Formula 1 can then react with H₂S (native, provided or generated from an H₂S donor) as described herein to generate hydrogen persulfide at the site of interest. An exemplary reaction depicting the linking of a compound of Formula 1 to an enzyme (lysozyme) is depicted below.

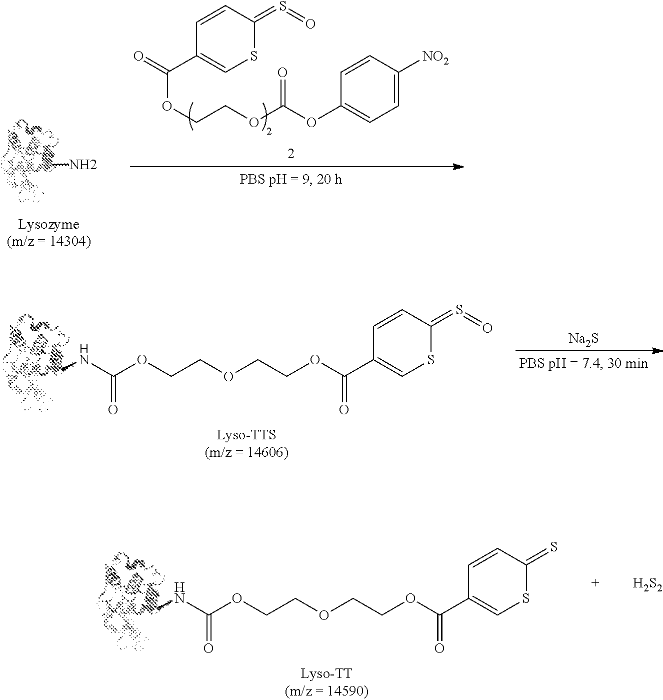

It is to be understood that a ligand, antibody or other delivery molecule, device or particle can be used to deliver the compound of the present disclosure to a location of interest, such as within an individual.

Exemplary receptor-ligand binding pairs include biotin, avidin, peptides, antibodies, folic acid, multivalent ligands, sigma-2 receptor and synthetic ligand SV119 and the like as are known to those of skill in the art.

Exemplary antibody-antigen/receptor binding pairs include anti-HER2 and HER2+, anti-cystathionine β-synthase (anti-CBS) and CBS, anti-cystahionine 7-lyase (anti-CSE) and CSE, anti-3-mercaptopyruvate sulfurtransferase (anti-3MPST), humanized IgG4 mAb and CD33, humanized IgG4 anti-CD22 and CD22, anti-IgM and IgM, anti-CD74 and CD74 and the like as are known to those of skill in the art.

Exemplary delivery molecules include peptides such as mitochondria-targeted peptides, organelle-targeting moieties such as triphenylphosphonium, morpholine, methylsulfonamide, and phenylsulfonamide, hydrogels such as biomimetic hyaluronic acid hydrogels, dipeptide hydrogels, micelles, polymer conjugates, and the like as are known to those of skill in the art.

Exemplary delivery devices include intranasal spray, sustained release gel or cream, capsule as are known to those of skill in the art.

Exemplary delivery particles include therapeutic nanoparticles (TNPs), gold nanoparticles (Au-NPs), gold nanorods, quantum dots, liposomal nanoparticles, nanofibers and the like as are known to those of skill in the art.

### VI. Exemplary Methods of Therapeutic Treatment

Aspects of the present disclosure include methods of treating an individual such as a human patient that would therapeutically benefit from generation of H₂S₂ at a therapeutic location within the individual. H₂S₂ is known to have biological applications. One or more exemplary compounds or pharmaceutically acceptable salts thereof as disclosed herein are administered to the individual at a target location wherein the one or more exemplary compounds or pharmaceutically acceptable salts thereof participate in a reaction to generate H₂S₂ in a therapeutically effective amount to treat an illness or condition associated with low levels of H₂S₂. As such, exemplary compounds as disclosed herein are used to treat diseases or conditions that can be cured or ameliorated by the generation of H₂S₂ and/or by an increase in the concentration of H₂S₂ at a location or environment of interest, such as the circulatory system of the individual, particular cells of the individual or any other location within the individual where generation of H₂S₂ is desired or useful for therapeutic purpose.

Exemplary compounds as disclosed herein may be within any suitable carrier system, such as a parenteral carrier system (including variations thereof such as the various specific injectable and infusible dosage forms) which may include one or more pharmaceutically suitable excipients, such as solvents like water and co-solvents, solubilizing compounds, wetting compounds, suspending compounds, thickening compounds, emulsifying compounds, chelating compounds, buffers, pH adjusters, antioxidants, reducing compounds, antimicrobial preservatives, bulking compounds, protectants, tonicity adjusters, and/or special additives.

One or more exemplary compounds as described herein may be included within a therapeutically effective dose that upon interaction with a biological material is sufficient to treat or prevent injury of a biological due to low levels of H₂S₂, whereby such dose may vary depending on the form of the compound, the biological material's condition and/or severity, the route of administration, the age of the biological material, and the like.

One or more exemplary compounds as described herein may be formulated into various forms for various routes of administration. Administration of a therapeutically effective dose will consist of one or more of the compounds as required. Administration of the compounds of the invention, or their pharmaceutically acceptable salts, in pure form or in an appropriate pharmaceutical composition, can be carried out via any of the accepted modes of administration of compounds for serving similar utilities. The pharmaceutical compositions of the invention can be prepared by combining at least one compound of the invention with an appropriate pharmaceutically acceptable carrier, diluent or excipient, and may be formulated. Pharmaceutical compositions of the invention are formulated so as to allow the one or more compounds disclosed herein to be bioavailable upon administration of the composition to a patient.

For example, a pharmaceutical composition of the invention is formulated to be compatible with its intended route of administration. Such pharmaceutical compositions may be administered by inhalation, transdermally, topically, orally, rectally, transmucosally, intestinally, parenterally, intramuscularly, subcutaneously, intravenously or other suitable methods that will be readily selected by the person of ordinary skill in the art, given the benefit of this disclosure. For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

In accordance with other examples, pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMPHOR ELTM (BASF, Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Oral compositions generally include an inert diluent or an edible carrier. Compound(s) as described herein can be enclosed in gelatin capsules or compressed into tablets. For the purpose of oral therapeutic administration, the compound(s) can be incorporated with excipients and used in the form of tablets, troches, or capsules. Oral compositions including the compound(s) can also be prepared using a fluid carrier for use as a mouthwash, wherein the compound(s) in the fluid carrier is applied orally and swished and expectorated or swallowed. Pharmaceutically compatible binding agents, and/or adjuvant materials can be included as part of the composition including the compound(s). The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The compositions of the invention typically contain the compounds in an amount that is sufficient to prevent or treat the condition/disease of interest. While any suitable amount of compound may be present in a composition, the amount generally ranges from about 1-99% by weight of all ingredients. One embodiment of the present disclosure involves a method of treating a non-human mammal or human which includes the step of administering a therapeutically and/or prophylactically effective amount of one or more compounds as described herein to a subject. As defined herein, a therapeutically and/or prophylactically effective amount of one or more compounds (i.e., an effective dosage) ranges from about 0.001 to 30 mg/kg body weight, from about 0.01 to 25 mg/kg body weight, from about 0.1 to 20 mg/kg body weight, from about 1 to 10 mg/kg, from about 2 to 9 mg/kg, from about 3 to 8 mg/kg, from about 4 to 7 mg/kg, or from about 5 to 6 mg/kg body weight. The skilled artisan will appreciate that certain factors may influence the dosage required to effectively treat a subject, including but not limited to the severity of the condition, previous treatments, the general health and/or age of the subject, and other diseases or conditions present. Treatment of a subject with a therapeutically and/or prophylactically effective amount of one or more compounds described herein can include a single treatment or can include a series of treatments. It will also be appreciated that the effective dosage of one or more compounds described herein may increase or decrease over the course of a particular treatment.

The therapeutic methods described herein can involve the steps of identifying a patient or subject with the disease or condition of interest, determining a suitable dosage of the compound(s), and administering the compound(s) to the subject/patient. Further, the methods may involve monitoring the response of the patient to the drug, and/or the outcome of treatment, and the modification of the original protocol as needed.

In accordance with certain other examples, kits for treating a subject are provided. A “kit” is a collection of parts forming the kit. In one example, the kit may include one or more compounds as described herein in a vial. In one example, the kit may include one or more H₂S donor compounds as described herein in a vial. In one example, the kit may include one or more H₂S donor compound conjugates as described herein in a vial. The kit may include a pharmaceutically acceptable carrier in a vial. The kit may include one or more compounds or H₂S donor compound conjugates as described as described herein in a pharmaceutically acceptable carrier in a vial. The kit may include one or more compounds attached to a delivery agent, such as a protein, receptor, ligand, antibody, delivery device or delivery particle as described as described herein in a pharmaceutically acceptable carrier in a vial. In an additional example, the kit may also include instructions for treating conditions characterized by a need to increase H₂S₂ in a subject by administering the one or more compounds described herein. In some examples, the kit may also comprise, e.g., a buffering agent, a preservative, or a protein stabilizing agent. In some examples, the kit may include an apparatus for administering the compound(s) described herein, such as a syringe or other apparatus. Other suitable components for including in the kit will be selected by the person of ordinary skill in the art, given the benefit of this disclosure.

Those of ordinary skill in the art recognize that, in some embodiments, the outcome may be a cure in that all disease symptoms disappear and do not return for at least a period of time. However, in other cases, administration may be ongoing in order to keep the condition or disease or symptoms to an acceptable level, or at least to a level that is lower than that experienced by the patient in the absence of treatment. Even a partial lessening of symptoms may be highly beneficial. In addition, the compounds of the invention may be administered in conjunction with the administration of other therapies or therapeutic compounds, e.g. other compounds suitable for treating the disease or condition which the compound is intended treat or prevent.

The following examples are set forth as being representative of the present disclosure. These examples are not to be construed as limiting the scope of the present disclosure as these and other equivalent embodiments will be apparent in view of the present disclosure, figures and accompanying claims.

### Example I

### Preparation and Reaction of TTS

The sulfine derivative of 2H-thiopyran-2-thione (TTS) was prepared from TT upon the treatment of m-CPBA as shown below. For ease of understanding, TTS is a shorthand reference and may refer to the substituted or unsubstituted compound of the present disclosure. 2H-thiopyran-2-thione (TTS) is then reacted with Na₂S as a proxy for H₂S, as is known to those of skill in the art, since the sodium salt dissolves in water to generate H₂S. In a mixed solvent system with PBS buffer (pH 7.4) and acetonitrile (1:1), the reaction between TTS and H₂S using Na₂S as the equivalent, produced TT in an almost quantitative yield (94%). The formation of H₂S₂ in this reaction was demonstrated by trapping H₂S₂ with phenyl 2-fluoro-5-nitrobenzoate, (see Liu, C.; et al., Rational design and bioimaging applications of highly selective fluorescent probes for hydrogen polysulfides. J. Am. Chem. Soc. 2014, 136, 7257-7260) which gave the desired product 5-nitro-3H-1,2-benzodithiol-3-one 1 in 80% yield.

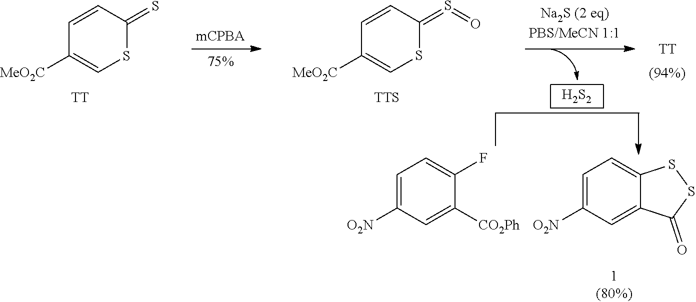

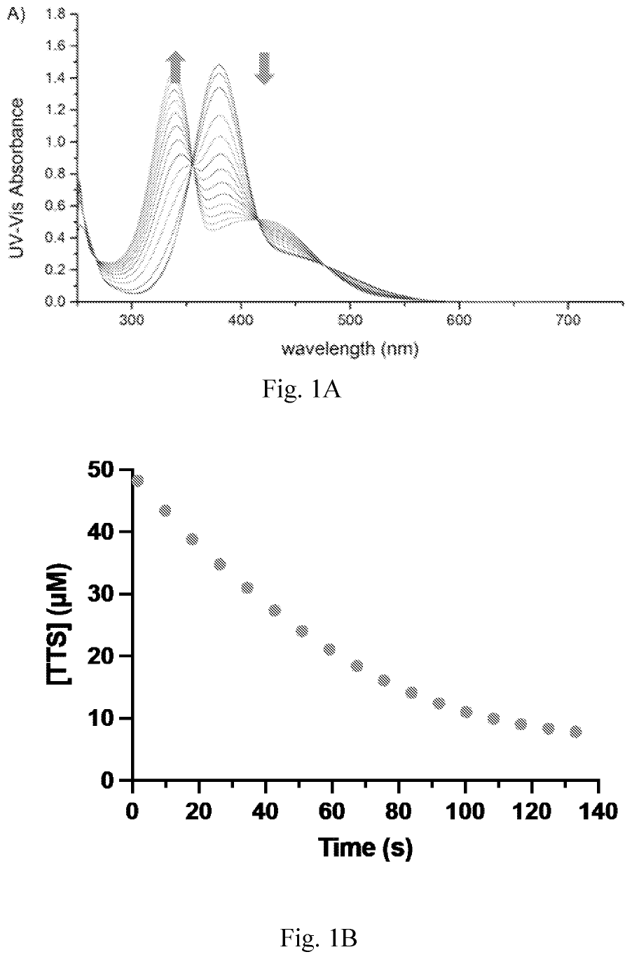

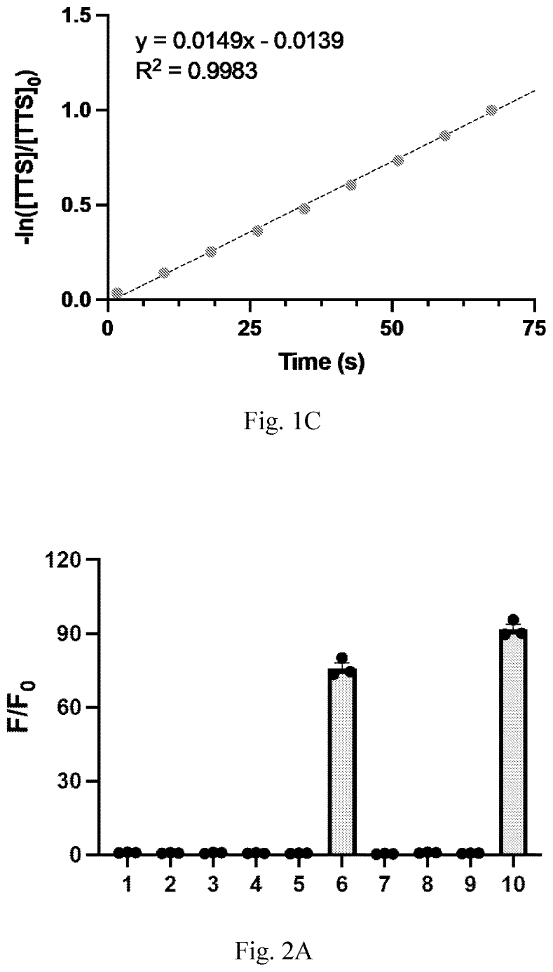

The reaction was monitored by UV-Vis insofar as TTS and TT possess different UV-Vis absorbance spectra at 380 and 339 nm, respectively. FIG. 1A depicts UV-Vis absorbance spectra changes of the reaction between TTS (100 μM) and H₂S (200 μM) over 1 min intervals. FIG. 1B depicts representative time-dependent [TTS] changes based on UV-Vis absorption changes at 380 nm of the reaction between TTS (50 μM) and Na₂S (5 mM). FIG. 1C depicts representative time-dependent −ln([TTS]/[TTS]₀) changes based on UV-Vis absorption changes at 380 nm of the reaction between TTS (50 μM) and Na₂S (5 mM). By measuring time-dependent spectra changes, the second-order rate constant, k₂, was calculated to be 2.7 μM⁻¹s⁻¹, indicating that this was a fast reaction comparable to some click reactions. See Kolb, H. C. et al., Click chemistry: diverse chemical function from a few good reactions. Angew. Chem. Int. Ed. 2001, 40, 2004-2021.

The TTS/H₂S reaction mechanism is presented in the scheme below, which was also studied by DFT (M06-2X) calculations.

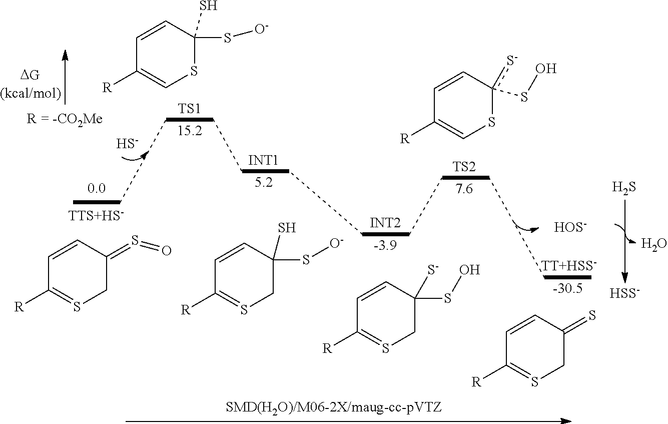

The reaction was initiated by a nucleophilic addition of HS⁻ to the thiocarbonyl carbon of TTS via TS1 to give intermediate INT1. This rate-determining step had an activation Gibbs free energy of 15.2 kcal/mol. Then, [1,4]-H shift on INT1 occurred to give thermodynamically favored INT2. A sulfur(0) species HOS⁻ would be released next via TS2 (ΔG^{‡}=11.5 kcal/mol), which could react with another molecule of H₂S to give HSS⁻, H₂O, and TT.

The mechanism was supported by an isotope labeling experiment shown in the scheme below, details of which are provided later herein.

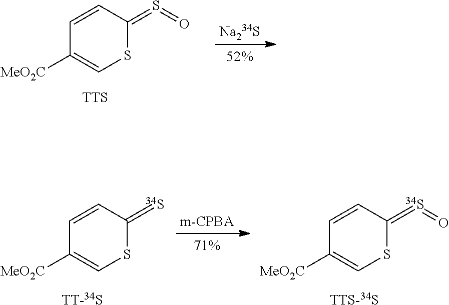

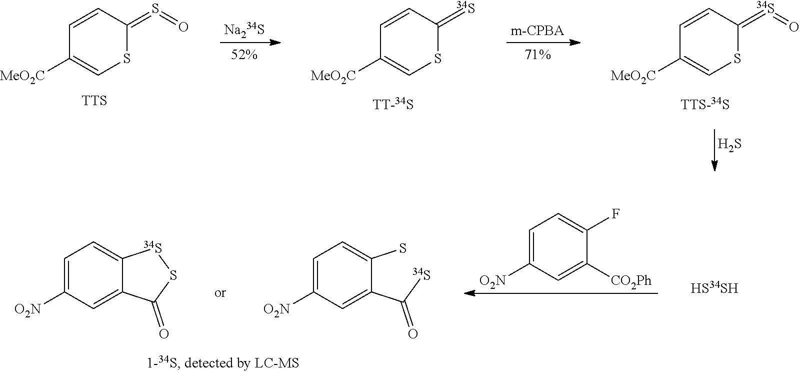

TTS was treated with ³⁴S-labeled Na₂S in PBS buffer (pH 7.4). As expected, ³⁴S-labeled product TT-³⁴S was obtained and characterized by MS analysis. It should be noted that TT-³⁴S could be converted into TTS-³⁴S upon oxidation. The present disclosure contemplates TTS-³⁴S cas a useful research reagent for the in-situ generation of ³⁴S-labeled H₂S₂, and the understanding of the biological functions of H₂S₂. 2-fluoro-5-nitrobenzoate was used to trap ³⁴S-labeled H₂S₂. ³⁴S-labeled product 1-³⁴S was obtained, albeit with the possibility of having two isomers.

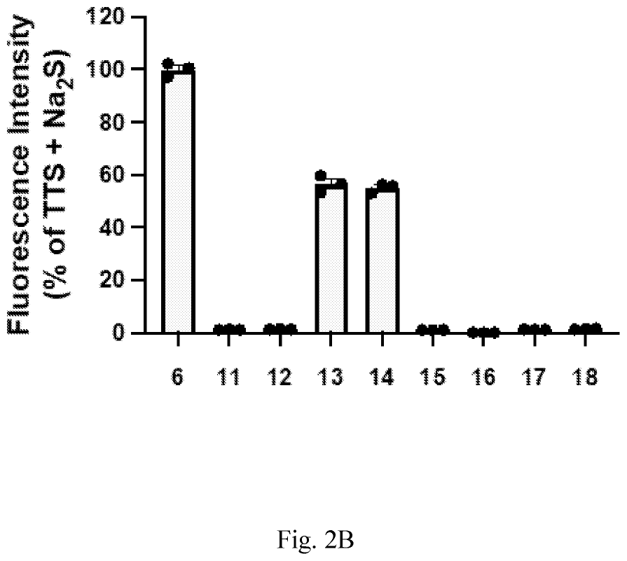

To further evaluate the specificity of TTS in converting H₂S to H₂S₂, SSP4 was used to monitor the formation of H₂S₂ in this reaction and the results were compared with a series of controls. Details of the SSP4 experiments are provided later herein. SSP4 is a well-established fluorescent sensor for sulfane sulfurs including H₂S₂. See Shieh, M. et al., Shining a light on SSP4: a comprehensive analysis and biological applications for the detection of sulfane sulfurs. Redox Biol. 2022, 56, 102433. As shown in FIG. 2A, only the co-existence of H₂S and TTS yielded significant fluorescence response. No fluorescence was observed in the cases of TTS/TT only, TTS+Cys, TT+H₂S, and TT+Cys. These results demonstrated that TTS could effectively convert H₂S to H₂S₂ without converting Cys to sulfane sulfurs. As shown in FIG. 2B, interference experiments with Cys or GSH were tested, and the presence of thiols led to decreased but still significant fluorescence. Without wishing to be bound by scientific theory, it is believed that thiols would compete with SSP4 to react with H₂S₂, thereby causing decreased signals. Taken together, these results demonstrated that TTS could effectively convert H₂S to H₂S₂. The results also showed that the byproduct TT would not cause unwanted reactions with Cys or H₂S.

### Example II

### Regenerating TTS from TT Using a Reactive Oxygen Species (ROS)

The reaction between TTS and H₂S is unique as it produces not only H₂S₂, but also TT, the precursor of TTS. TTS was demonstrated to be regenerable upon reaction with an oxidant, which supports TTS being regenerated in a biological system where an oxidant is present.

TT was treated with H₂O₂, the most prevalent oxidant in biology. Stock solutions for hydrogen peroxide (H₂O₂), t-butyl hydroperoxide (^{t}BuOOH), peracetic acid (AcOOH), and sodium hypochlorite (NaOCl), were prepared in deionized water. H₂O₂ was diluted from a commercially available solution (30%). The concentration of H₂O₂ was determined using the absorption at 240 nm (ε=43.6 μM⁻¹ cm⁻¹). ^{t}BuOOH was diluted from commercially available solutions (70%). AcOOH was diluted from commercially available solutions (32%). NaOCl was diluted from commercially available solution containing 11-15% available chlorine. The concentration of HOCl was determined using the absorption at 292 nm (ε=391 μM⁻¹ cm⁻¹).

TT was dissolved in DMSO to afford a 10 mM stock solution. For H₂O₂, ^{t}BuOOH and AcOOH, substrate TT (10 μM) and ROS (1 mM) were mixed in PBS buffer, pH=7.4 with DMSO (1%) under room temperature. For NaClO, TT (100 μM) and NaClO (1 mM) were mixed in PBS buffer, pH=7.4 with DMSO (1%) under room temperature. The rate of production of TTS was detected by UV-Vis spectrum (λ=339 nm) based on the decrease of TT. A pseudo-first order reaction was assumed; the second-order k constant (k₂) was calculated using the method described below.

To determine [TT] at each time point:

$$A = {ε}_{TTS} \times [ TTS ] + {ε}_{TT} \times [ TT ]$$

Considering the starting concentration of [TT] was 10 μM or 100 μM, [TTS]+[TT]=10 or 100 μM, let the starting concentration of [TT]=x.

$$A = {ε}_{TTS} \times ( x - [ TT ] ) + {ε}_{TT} \times ( [ TT ] )$$

$${ε}_{TTS ( 339 nm )} = 0.0 \; 029 {μM}^{- 1} {cm}^{- 1} and {ε}_{T T ( 339 nm )} = 0.0 \; 142 {μM}^{- 1} {cm}^{- 1}$$

$$\frac{A - 0.0029 x}{0.0113} = [ TT ]$$

$${k}_{obs} = k [ ROS ] , where [ ROS ] = 0.001 M$$

$${k}_{obs} t = - ln \frac{[ TT ]}{{[ TT ]}_{0}} , {where [ TT ]}_{0} = 10 or 100 μM$$

To an NMR tube, the solution of TT (0.2 mmol, final concentration is 20 mM) in CD₃CN/D₂O (1:1) was added, followed by adding H₂O₂ (10 μM, 20 μM). The NMR tube was shacked immediately and incubated at room temperature for 0 min, 30 min, 3 h, and 24 h.

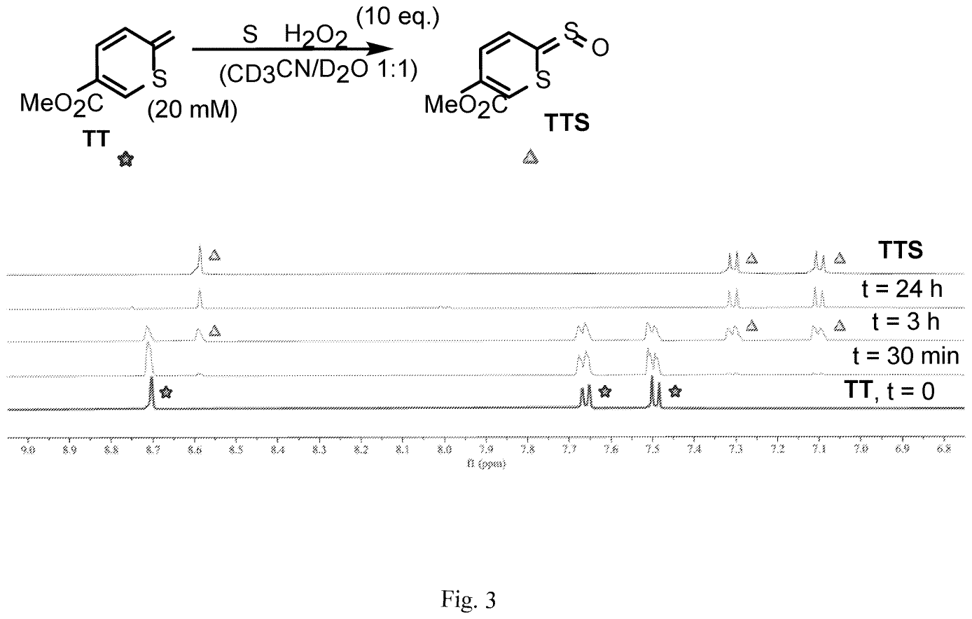

As shown in FIG. 3, the NMR spectrum showed the reaction occurred within 3 h and completed in 24 h. The transformation from TT to TTS was clean. The progress of the reaction was monitored by UV-Vis spectra and the reaction rate constant (k₂) was measured to be 0.1 μM⁻¹s⁻¹. The oxidation of TT by several other oxidants including t-BuOOH, AcOOH, and OCl⁻ was measured and the TT oxidation rate constants k₂ are summarized in Table 1. ^{a}The rate constant was estimated.

TABLE 1

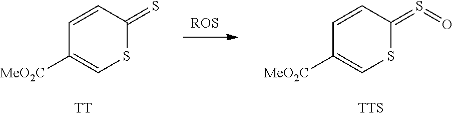

| ROS | k₂ |
|---|---|
| H₂O₂ | 0.1 M⁻¹ s⁻¹ |
| ^{t}BuOOH | 1.0 x 10⁻² M⁻¹ s⁻¹ |
| AcOOH | 13.9 M⁻¹ s⁻¹ |
| OCl⁻ | >1.0 × 10³ M⁻¹ s^{-1 a} |

### Example III

### Cellular Environments

To demonstrate that the reaction between TTS and H₂S could proceed under cellular environments, live cell imaging studies were carried out. HeLa cells were seeded at ~7000 cells/well in a 96-well clear flat-bottomed BioLite plate (ThermoFisher Scientific, #130188) in DMEM/F12 (1:1) (Gibco, Invitrogen, #11330-032) medium supplemented with 10% fetal bovine serum (FBS) at 37° C., 5% CO₂ overnight.

Preparation before cell treatment: Na₂S and Na₂S₂ stocks were prepared at 10 mM in sterile 1×PBS. TTS and TT were prepared at 10 mM concentrations in DMSO. They were then diluted to 50 μM concentrations in serum-free Fluorobrite DMEM in individual Eppendorf tubes (0.5% DMSO). Na₂S (final concentration=100 μM) was added to the Eppendorf tube containing Fluorobrite DMEM and TTS or TT immediately before being added to the cells. Compounds were mixed by pipetting up and down prior to cell addition.

Cell treatment: Culturing media was aspirated from all wells, and cells were washed twice with 1×PBS. Compounds were added to cells (200 μL/well) and incubated for 30 min at 37° C., 5% CO₂. While cells were incubating, SSP4 treatment was prepared by first adding 5 mM CTAB in ethanol to Fluorobrite DMEM followed by 6.125 mM SSP4 in DMSO to final concentrations of 100 μM and 5 μM, respectively, in a tube. The final volume was 5 mL (0.08% DMSO). Compounds were mixed by pipetting up and down prior to cell addition. After incubation, media was aspirated, and cells were washed twice with 1×PBS. SSP4 treatment was added to all wells (100 μL/well), and cells were incubated for 30 min at 37° C., 5% CO₂ for 30 min. Media was then aspirated, and cells were washed three times with 1×PBS. Fluorobrite DMEM was added to all wells before imaging on the Keyence All-in-One Fluorescence Microscope (BZ-X810) (excitation: 470/40 nm; emission: 525/50 nm).

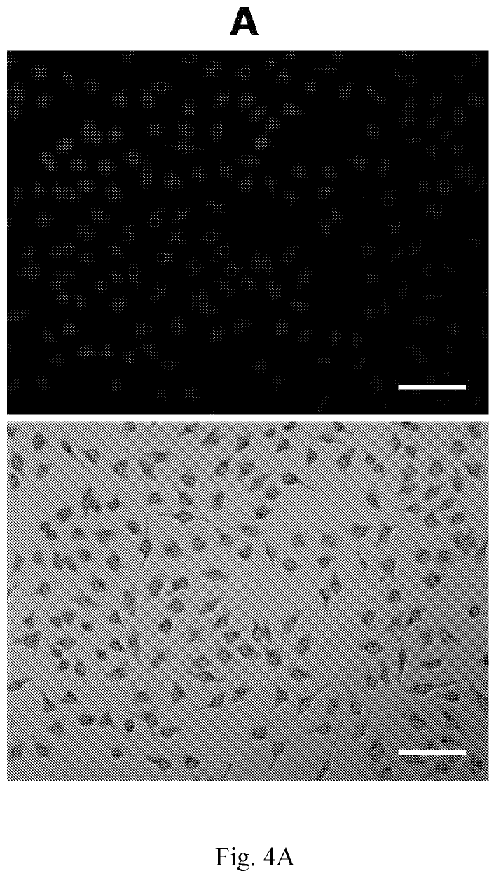

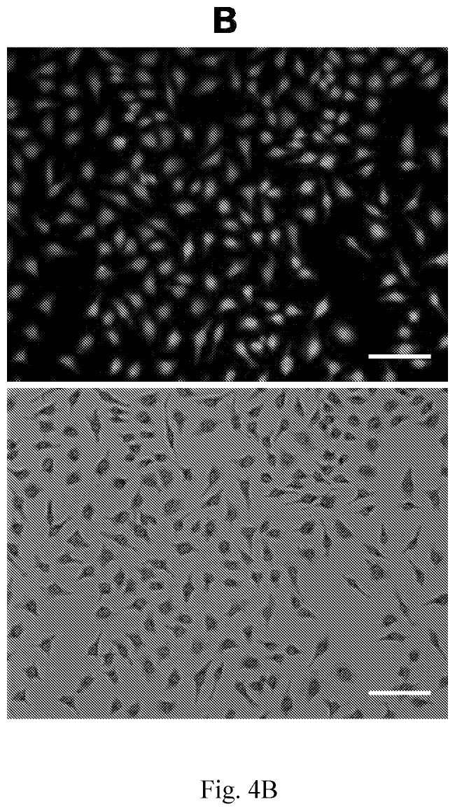

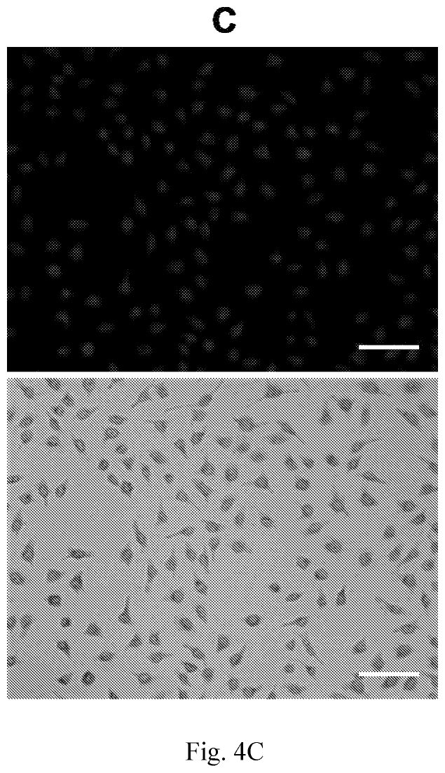

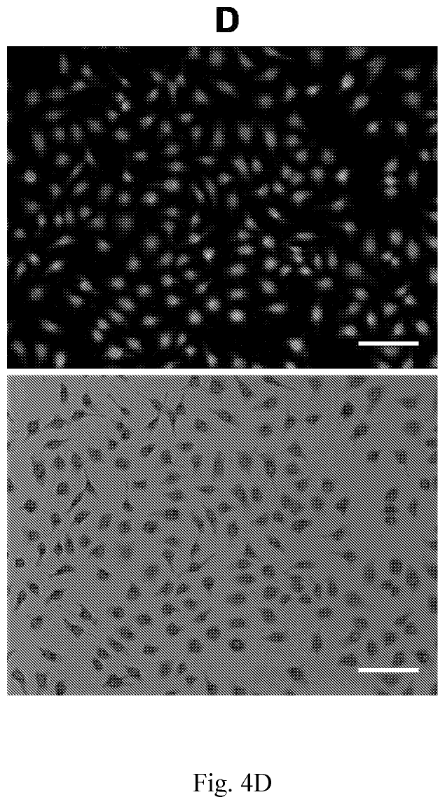

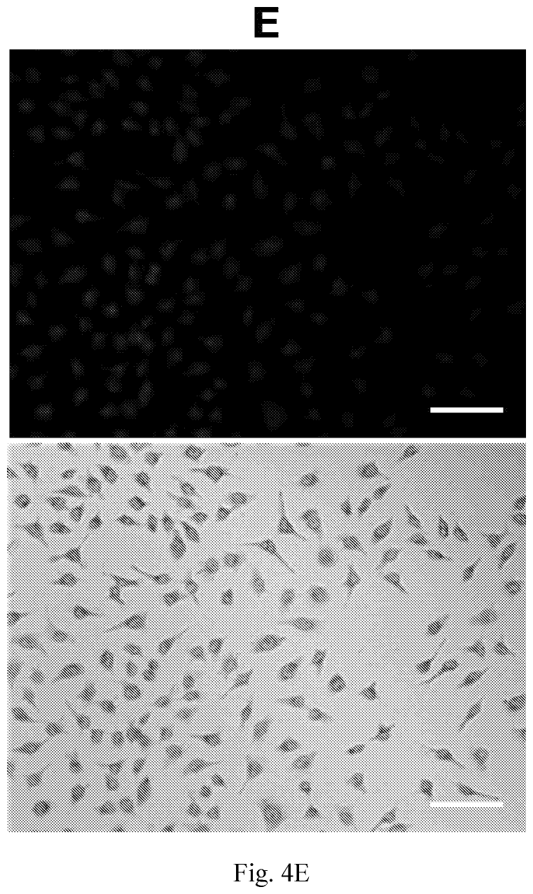

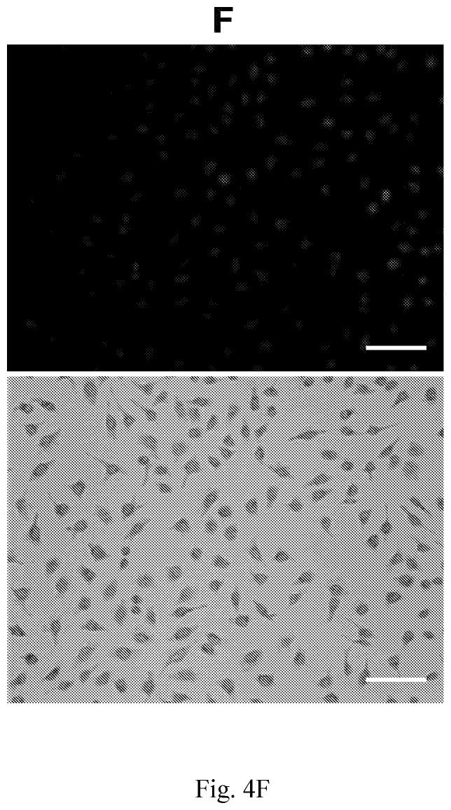

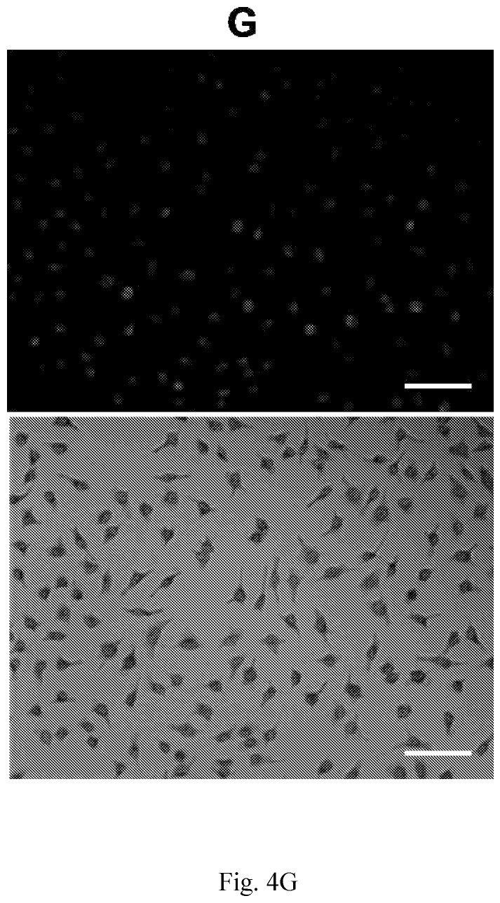

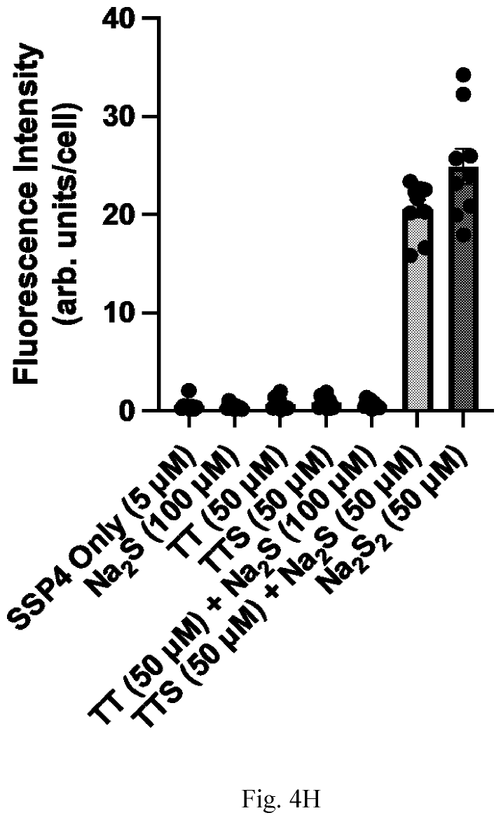

As shown in FIGS. 4(A)-(D), HeLa cells were treated with TTS, TTS+H₂S, or TT for 30 min before the fluorescent sensor SSP4 was applied to the cells to monitor the formation of H₂S₂. Significant green fluorescence in cells with both TTS and H₂S was observed (FIG. 4B). The other two treatments (TTS only or TT only) did not show obvious fluorescence. Na₂S₂ was used as the positive control in this study (FIG. 4D). Other control studies including TT+H₂S, H₂S only, and SSP4 only were also performed. Obvious fluorescence in these samples was not observed (FIG. 4E~G). These results demonstrated that TTS could effectively convert H₂S to H₂S₂ in complex biological systems. FIG. 4H is a graph of fluorescence intensities of SSP4 staining. Data are mean values ±SEM calculated using ImageJ. (n=9, where three different images were obtained from each of three samples and quantified).

### Example IV

### Protein Conjugates

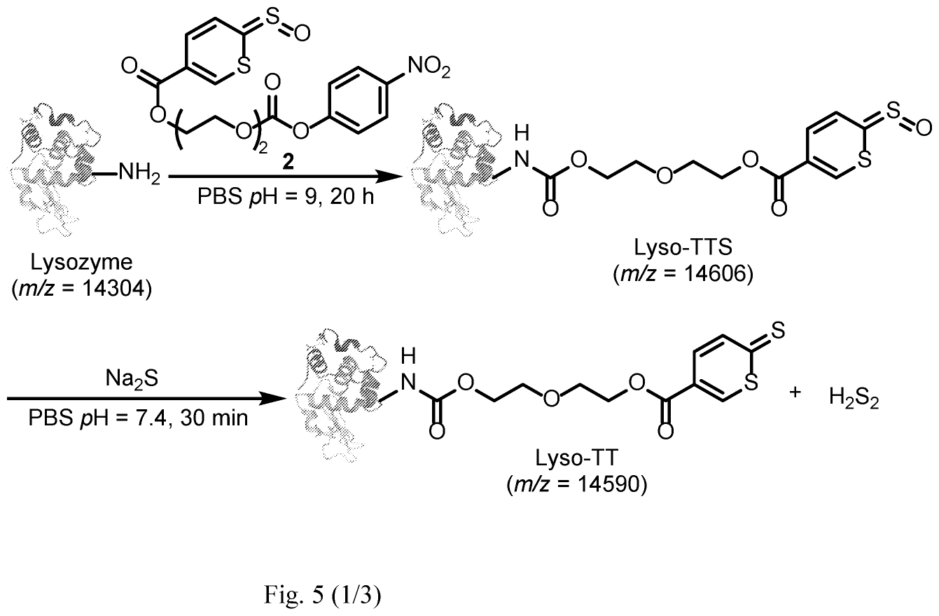

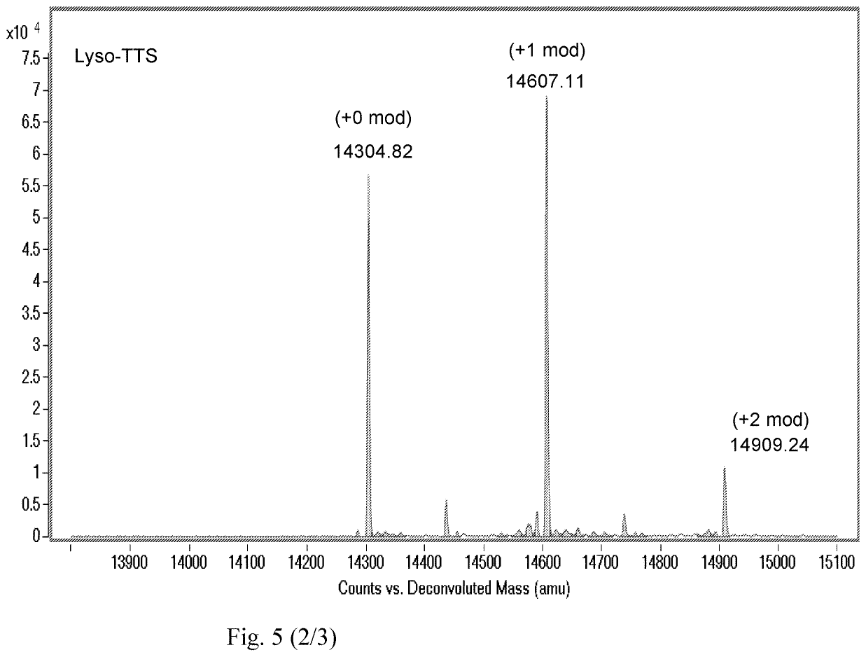

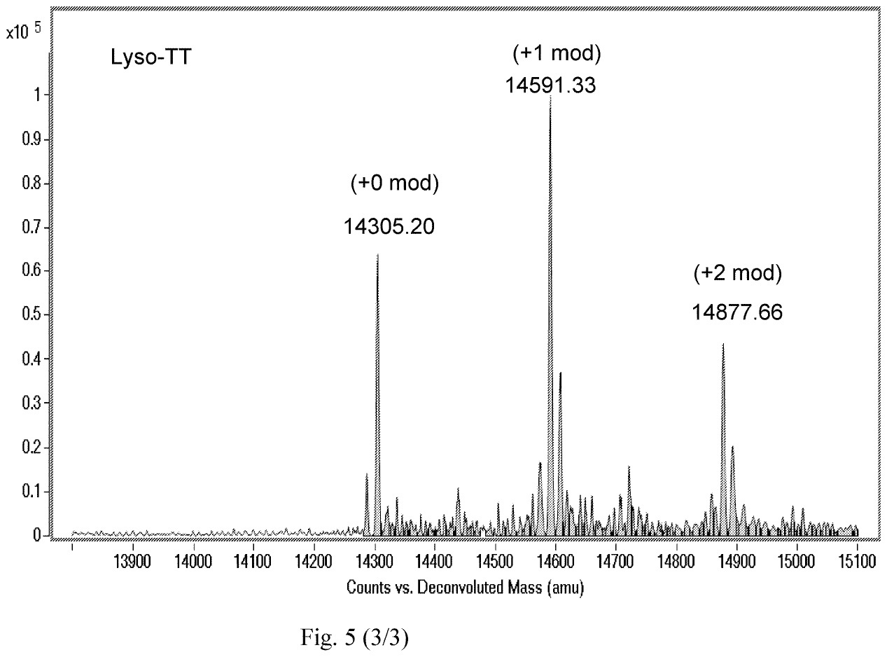

Since the reaction between TTS and H₂S is bio-orthogonal, the present disclosure contemplates the delivery of TTS to certain cellular locations or conjugated with certain biomolecules to achieve specific/localized H₂S to H₂S₂ transformation. A TTS-conjugated p-nitrophenol carbonate 2 was prepared and treated with a model protein-lysozyme C. As shown in FIG. 5, the TTS moiety was successfully linked to the protein. When the modified protein (Lyso-TTS) was then treated with H₂S, the desired product Lyso-TT was obtained (as demonstrated by MS). The formation of H₂S₂ in this reaction was also confirmed. These results further demonstrated the stability and applicability of TTS in biological systems. For example, protein-bound TTS can be engineered for controlled H₂S₂ delivery or controlled induction of protein modifications via protein-protein interactions.

### Example V

### Synthesis of an Exemplary Compound of Formula 1

An exemplary compound 2H-thiopyran-2-thione (TT), methyl 2-thioxo-2H-thiopyran-5-carboxylate, is a known compound and was prepared according to the method described in Defoina, A. et al., Helv. Chim. Acta, 1985, 68, 1998 and was characterized using NMR as follows. ¹H NMR (400 MHz, CDCl₃) δ 8.48 (dd, J=1.8, 1.2 Hz, 1H), 7.58 (dd, J=10.4, 1.8 Hz, 1H), 7.44 (dd, J=10.4, 1.2 Hz, 1H), 3.91 (s, 3H).

The sulfine derivative was prepared according to the reaction below and characterized as follows.

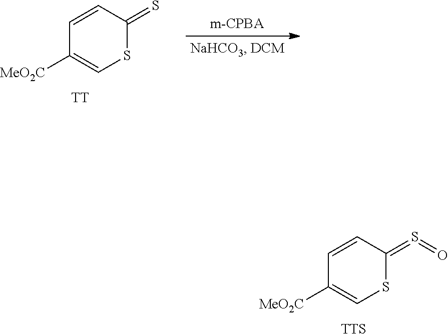

To a stirred solution of methyl 2-thioxo-2H-thiopyran-5-carboxylate (exemplary TT) (186 mg, 1.0 mmol) in DCM (10 mL), NaHCO₃ (84 mg, 5.0 mmol, 5.0 eq) and m-CPBA (70%, 246 mg, 1.0 mmol, 1.0 eq) were added. After being stirred at room temperature for 30 min, the reaction was quenched by Sat. NaHCO₃ solution and extracted by ethyl acetate (EA). Combined organic layers were washed with brine and dried by Na₂SO₄. The solvent was removed under reduced pressure. The crude material was purified by flash column chromatography (HEX/EA=5/1-3/1) to yield TTS (153 mg, yield=76%) as a dark red powder. R_{f}: 0.3 (HEX/EA=2/1), m.p.=130-131° C. ¹H NMR (600 MHz, CD₂Cl₂) δ 8.32 (dd, J=1.2, 1.2 Hz, 1H), 7.06 (dd, J=10.5, 1.2 Hz, 1H), 6.93 (dd, J=10.5, 1.2 Hz, 1H), 3.85 (s, 3H). ¹³C NMR (151 MHz, CD₂Cl₂) δ 184.7, 163.3, 139.1, 126.8, 124.8, 120.2, 53.0. HRMS (ESI, m/z): [M+H]⁺ calculated for C₇H₇O₃S₂⁺: 202.9837. found: 202.9836.

The unsubstituted sulfine derivative referred to herein as TTS-0 was prepared and characterized as follows.

### Synthesis of TTS-0

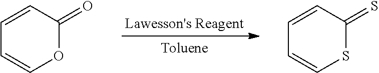 [SMILES: O=c1cccco1 ; S=c1ccccs1]

To an oven-dried round bottom flask with a magnetic stir bar was added 2H-pyran-2-one (719 mg, 7.49 mmol), Lawesson's Reagent (3.80 g, 9.40 mmol), and toluene (100 mL). The flask was sealed with a rubber septum, evacuated and backfilled with argon. The reaction mixture was heated to 100° C. for 72 h. After cooling to room temperature, the reaction mixture was concentrated in vacuo. The obtained residue was purified via flash chromatography (100% toluene) to yield 2H-thiopyran-2-thione (126 mg, yield=13.3%) as a red solid.

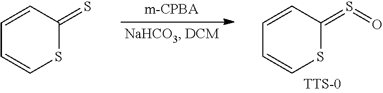 [SMILES: S=c1ccccs1 ; O=S=c1ccccs1]

To an oven-dried round bottom flask with a magnetic stir bar was added 2H-thiopyran-2-thione (97 mg, 0.75 mmol), m-CPBA (70%, 175 mg, 0.71 mmol), NaHCO₃ (315 mg, 3.75 mmol), and DCM (15 mL). After being stirred at room temperature for 30 min, an aqueous solution of saturated NaHCO₃ was added. The aqueous solution was extracted with EtOAc. The combined organic layer was washed with brine, dried over Na₂SO₄, filtered and concentrated in vacuo. The resulting residue was purified via flash chromatography (0-25% EtOAc/Hexanes) to yield (2H-thiopyran-2-ylidene)-λ⁴-sulfanone (65 mg, yield=63.6%) as a dark red solid.

TTS-0: ¹H NMR (600 MHz, DMSO-d₆) δ 7.86 (d, J=9.5 Hz, 1H), 7.37 (d, J=10.2 Hz, 1H), 6.91 (dd, J=9.5, 6.8 Hz, 1H), 6.67 (dd, J=10.1, 6.9 Hz, 1H). ¹³C NMR (151 MHz, DMSO-d₆) δ 186.94, 132.09, 126.58, 123.56, 119.55. HRMS (ESI, m/z): [M]⁺ calculated for C₅H₄OS₂: 143.9704. found: 143.9713.

The following compounds for use as disclosed herein were prepare and characterized as follows.

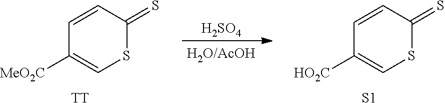

S1: To a stirred solution of methyl 2-thioxo-2H-thiopyran-5-carboxylate TT (117 mg, 0.63 mmol) in acetic acid (5 mL), H₂SO₄ (0.75 mL, 50% v/v) was added. After being stirred at 100° C. for 5 h, the reaction was quenched by H₂O and extracted with DCM. Combined organic layers were washed by brine and dried by Na₂SO₄. The solvent was removed by rotary evaporator. The crude material was purified by flash column chromatography (EA with 1% AcOH) to yield S1 (84 mg, yield=78%) as a red powder.

S1: R_{f}: 0.05 (EA), m.p.>200° C. ¹H NMR (600 MHz, CD₃OD) δ 8.72 (dd, J=1.9, 1.1 Hz, 1H), 7.67 (dd, J=10.3, 1.9 Hz, 1H), 7.46 (dd, J=10.3, 1.1 Hz, 1H). ¹³C NMR (151 MHz, CD₃OD) δ 207.8, 165.3, 150.1, 140.3, 133.0, 127.3. HRMS (ESI, m/z): [M+H]⁺ calculated for C₆H₅O₂S₂⁺: 172.9731. found: 172.9718.

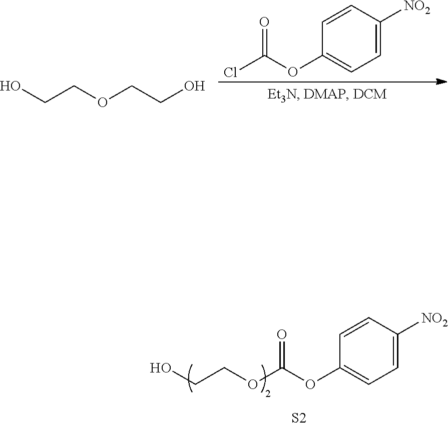

S2: To a stirred solution of diethylene glycol (1.92 mL, 20 mmol, 2.0 eq) in DCM (70 mL), a mixture of 4-nitrophenyl carbonochloridate (2.02 g, 10 mmol, 1.0 eq), Et₃N (2.76 mL, 20 mmol, 2.0 eq), DMAP (61 mg, 0.5 mmol, cat.) and DCM (30 mL) was added dropwise at 0° C. The mixture was stirred at room temperature for 30 min and then concentrated under reduced pressure. The residue was diluted with H₂O and extracted with ethyl acetate (EA). Combined organic layers were washed by brine and dried by Na₂SO₄. The solvent was removed by rotary evaporator. The crude material was purified by flash column chromatography (hexane (HEX)/EA=1/1-1/2) to yield S2 (365 mg, yield=13%) as a colorless oil.

S2: R_{f}: 0.2 (EA). ¹H NMR (600 MHz, CDCl₃) δ 8.28 (d, J=8.7 Hz, 2H), 7.39 (d, J=8.7 Hz, 2H), 4.46 (t, J=4.5 Hz, 2H), 3.82 (t, J=4.5 Hz, 2H), 3.78 (t, J=4.5 Hz, 2H), 3.66 (t, J=4.5 Hz, 2H), 1.80 (brs, 1H). ¹³C NMR (151 MHz, CDCl₃) δ 155.6, 152.7, 145.6, 125.5, 121.9, 72.6, 68.8, 68.3, 61.9. HRMS (ESI, m/z): [M+H]⁺ calculated for C₁₁H₁₄NO₄⁺: 272.0770. found: 272.0766.

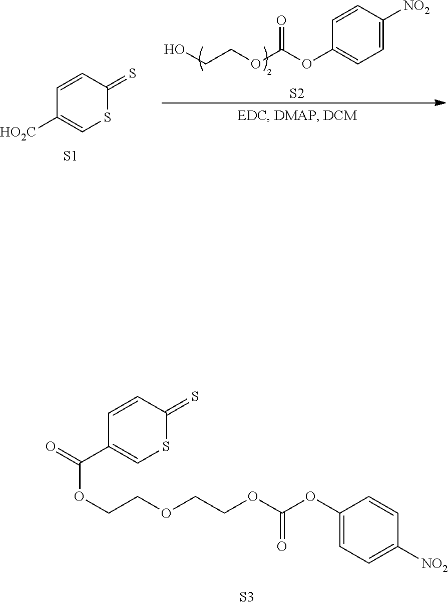

S3: To a stirred solution of S1 (181 mg, 1.05 mmol, 1.05 eq), S2 (271 mg, 1.0 mmol, 1.0 eq), DMAP (12 mg, 0.1 mmol, 0.1 eq) in DCM (10 mL), EDC (288 mg, 1.5 mmol, 1.5 eq) was added. The mixture was stirred at room temperature for 24 h under Ar atmosphere. Then, the reaction was quenched by H₂O and extracted by EA. Combined organic layers were washed by brine and dried by Na₂SO₄. The solvent was removed by rotary evaporator. The crude material was purified by flash column chromatography (HEX/EA=3/1-2/1) to yield S3 (180.5 mg, yield=42%) as a red powder, which was used directly for the next reaction below to make compound 2.

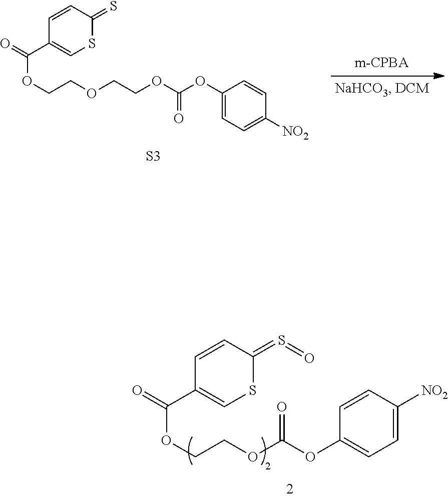

Compound 2: To a stirred solution of S3 (165 mg, 0.39 mmol) in DCM (5 mL), NaHCO₃ (164 mg, 1.95 mmol, 5.0 eq) and m-CPBA (70%, 123 mg, 0.5 mmol, 1.3 eq) were added. The mixture was stirred at room temperature for 10 min. Then, the reaction was quenched by saturated NaHCO₃ solution and extracted by EA. Combined organic layers were washed by brine and dried by Na₂SO₄. The solvent was removed by rotary evaporator. The crude material was purified by flash column chromatography (HEX/EA=2/1-1/2) to yield 2 (105 mg, yield=61%) as a dark red powder.

2: R_{f}: 0.5 (EA), m.p.=81-83° C. ¹H NMR (600 MHz, CD₂Cl₂) δ 8.35 (s, 1H), 8.26 (d, J=9.2 Hz, 2H), 7.39 (d, J=9.2 Hz, 2H), 7.04 (d, J=10.5 Hz, 1H), 6.93 (d, J=10.5 Hz, 1H), 4.47-4.27 (m, 4H), 4.06-3.51 (m, 4H). ¹³C NMR (151 MHz, CD₂Cl₂) δ 184.6, 162.8, 155.9, 152.9, 145.9, 139.5, 126.7, 125.7, 124.7, 122.2, 120.2, 69.3, 69.0, 68.6, 65.0. HRMS (ESI, m/z): [M+H]⁺ calculated for C₁₇H₁₆NO₉S₂⁺: 442.0266. found: 442.0262.

TAGDD-6 is a known compound and was prepared according to the method described Zhao, Y. et al., Org. Lett. 2014, 16, 4536.

TAGDD-6: ¹H NMR (400 MHz, CDCl₃) δ 7.18 (t, J=9.0 Hz, 1H), 6.99 (d, J=9.0 Hz, 1H), 6.94-6.86 (m, 1H), 6.75-6.70 (m, 1H), 6.19 (s, 1H), 2.32 (s, 6H).

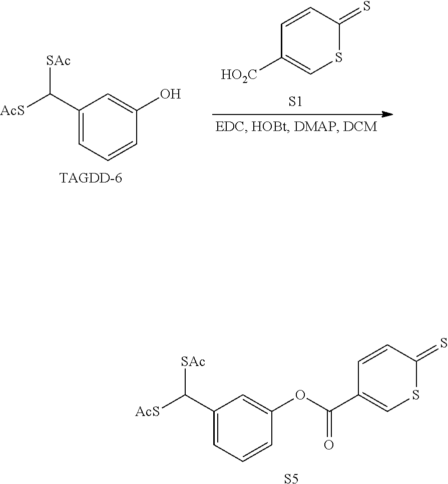

S5: To a stirred solution of TAGDD-6 (166 mg, 0.65 mmol, 1.05 eq), S1 (107 mg, 0.62 mmol, 1.0 eq), DMAP (17 mg, 0.14 mmol, 0.2 eq), HOBt (142 mg, 0.93 mmol, 1.5 eq) in DCM (15 mL), EDC (178 mg, 0.93 mmol, 1.5 eq) was added. After being stirred at room temperature for 24 h under Ar atmosphere, the reaction was quenched by H₂O and extracted by EA. Combined organic layers were washed by brine and dried by Na₂SO₄. The solvent was removed by rotary evaporator. The crude material was purified by flash column chromatography (HEX/EA=5/1-3/1) to yield S5 (163 mg, yield=64%) as a red powder, which was used directly for the next reaction below to make compound 3.

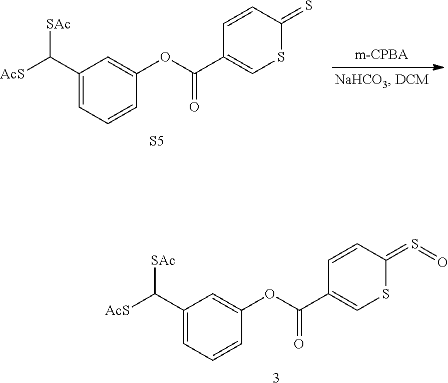

Compound 3: To a stirred solution of S5 (163 mg, 0.4 mmol) in DCM (5 mL), NaHCO₃ (168 mg, 2.0 mmol, 2.0 eq) and m-CPBA (70%, 78 mg, 0.4 mmol, 1.0 eq) were added. After being stirred at room temperature for 10 min, the reaction was quenched by saturated NaHCO₃ solution and extracted by EA. Combined organic layers were washed by brine and dried by Na₂SO₄. The solvent was removed by rotary evaporator. The crude material was purified by flash column chromatography (HEX/EA=3/1-2/1) to yield 3 (119 mg, yield=70%) as a dark red powder. 3: R_{f}: 0.2 (HEX/EA=1/1), m.p.=97-98° C. ¹H NMR (600 MHz, CD₂Cl₂) δ 8.53 (dd, J=1.2, 1.2 Hz, 1H), 7.41-7.34 (m, 2H), 7.28-7.23 (m, 1H), 7.12 (dd, J=10.5, 1.2 Hz, 1H), 7.10-7.08 (m, 1H), 7.01 (dd, J=10.5, 1.2 Hz, 1H), 6.20 (s, 1H), 2.32 (s, 6H). ¹³C NMR (151 MHz, CD₂Cl₂) δ 192.8, 184.2, 161.4, 150.8, 142.1, 140.8, 130.2, 126.12, 126.08, 124.5, 121.7, 121.3, 120.5, 47.7, 30.3. HRMS (ESI, m/z): [M+H]⁺ calculated for C₁₇H₁₅O₅S₄⁺: 426.9802. found: 426.9797.

### Example VI

### Reaction of TT with Na₂S as an Equivalent of H₂S

The following reaction was carried out.

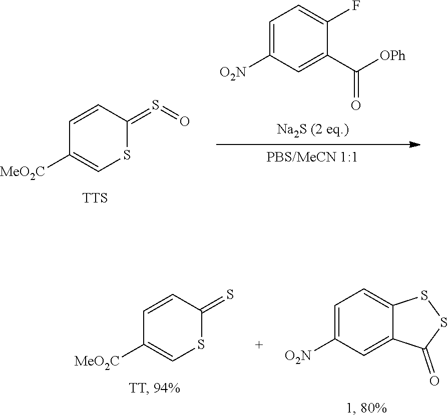

To a stirred solution of TTS (19.6 mg, 0.1 mmol) in MeCN (5 mL), Na₂S·9H₂O (48 mg, 0.2 mmol, 2.0 eq) in PBS buffer (5×pH=7.4, 6 mL) was added dropwise. After being stirred at room temperature for 1 h, phenyl 2-fluoro-5-nitrobenzoate (52 mg, 0.2 mmol, in 1 mL MeCN) was added dropwise to demonstrate production of H₂S₂. The reaction was stirred for another 1 h and then quenched by H₂O and extracted by EA. Combined organic layers were washed by brine and dried by Na₂SO₄. The solvent was removed by rotary evaporator. The crude material was purified by Perp TLC (HEX/EA=5/1) to yield TT (17.0 mg, yield=94%) and 1 (16.5 mg, yield=80%). The ¹H-NMR of the products were consistent with reported literatures.

TT: ¹H NMR (400 MHz, CDCl₃) δ 8.49 (dd, J=1.9, 1.1 Hz, 1H), 7.61 (dd, J=10.3, 1.9 Hz, 1H), 7.44 (dd, J=10.3, 1.1 Hz, 1H), 3.91 (s, 3H).

1: ¹H NMR (600 MHz, CDCl₃) δ 8.79 (d, J=2.3 Hz, 1H), 8.49 (dd, J=8.9, 2.3 Hz, 1H), 7.78 (d, J=8.9 Hz, 1H).

### Example VII

### Persulfidating Proteins Using TTS and H₂S to Generate H₂S₂

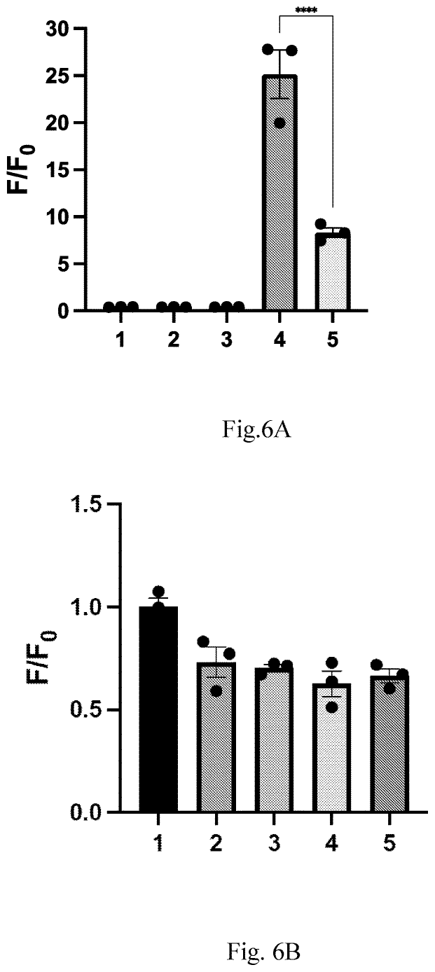

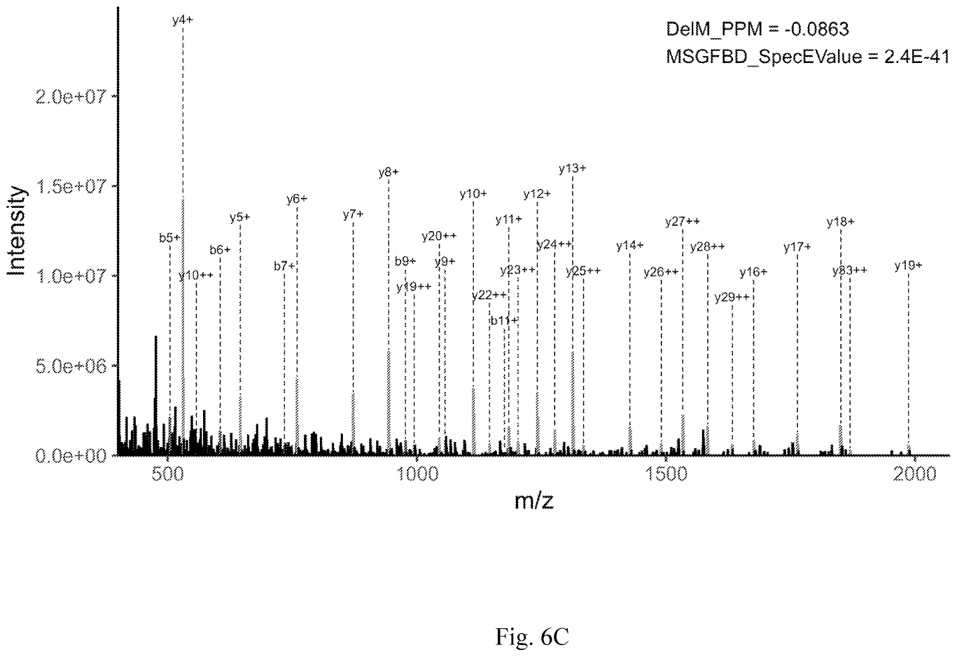

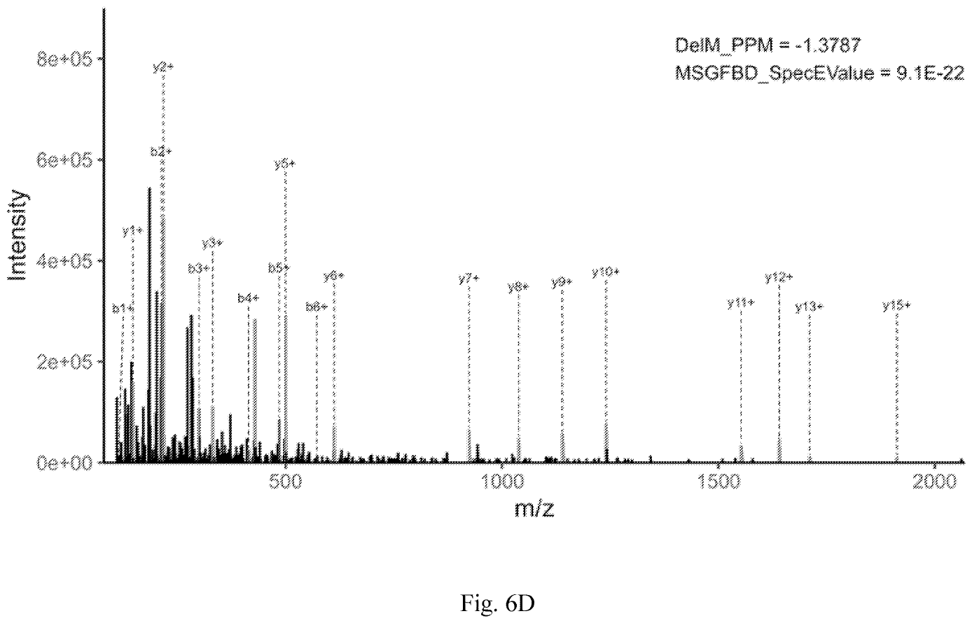

One of the primary functions of H₂S₂ is to induce protein S-persulfidation. Existing methods treat proteins with Na₂S₂ directly to obtain the persulfidated proteins. The present disclosure contemplates the use of TTS and H₂S to persulfidate proteins as a comparison with Na₂S₂. GAPDH was used as the protein model and its persulfidation was evaluated by SSP4 using a protocol described in Shieh, M. et al., Shining a light on SSP4: a comprehensive analysis and biological applications for the detection of sulfane sulfurs. Redox Biol. 2022, 56, 102433 and Ni, X. et al., A sweet H₂S/H₂O₂ dual release system and specific protein S-persulfidation mediated by thioglucose/glucose oxidase. J. Am. Chem. Soc. 2021, 143, 13325-13332. Reduced GAPDH (100 μM) was individually treated with 10 eq. of TTS+Na₂S or Na₂S₂, as well as control reagents (TTS or Na₂S only). The protein samples were then purified using Zeba Spin Desalting Columns (7K MWCO), diluted (to 10 μM), and treated with SSP4 (5 μM). Fluorescence signals were recorded to reflect persulfidation levels. As shown in FIG. 6, control samples did not give any notable fluorescence. Samples treated with TTS+Na₂S or Na₂S₂ yielded strong fluorescence while statistical analysis revealed that the combination of TTS and Na₂S gave significantly higher signals than Na₂S₂. To demonstrate that the observed fluorescence signals were not due to the insufficient removal of Na₂S₂ in the desalting step, control studies using —SH blocked GAPDH and small molecules only samples under the same conditions were carried out. These samples depicted in FIG. 6B did not give any fluorescence. To provide further evidence of persulfidation in the GAPDH system, TTS+Na₂S-treated GAPDH was alkylated with HPE-IAM to stabilize the protein persulfides. The protein was then digested with trypsin, and the peptides were subjected to LC-MS/MS analysis which indicated successful protein persulfidation as shown in FIG. 6CD. Together, these results confirm the efficiency and advantage of using TTS and H₂S (the TTS booster system) to generate H₂S₂ in protein persulfidation.

### Example VIII

### Isotope Labelling Experiments

Isotope labelling experiments as described herein were carried out using the following reaction.

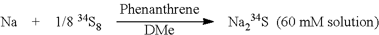

To a stirred suspension of ³⁴S₈ (31 mg, 0.91 mmol), Phenanthrene (102 mg, 0.57 mmol) in dry DME (5 mL), sodium metal (112 mg, 4.87 mmol) was added under Ar atmosphere. The solution turned dark green when being heated. After being stirred at 80° C. for 16 h, excess sodium metal and the solvent were removed. The residue was partially dissolved in H₂O. The undissolved organic compound was removed by filtration. The concentration of Na₂³⁴S solution was analyzed by methylene blue method compared with standard Na₂S sample (~60 mM).

Isotope labelling experiments as described herein were carried out using the following reaction.

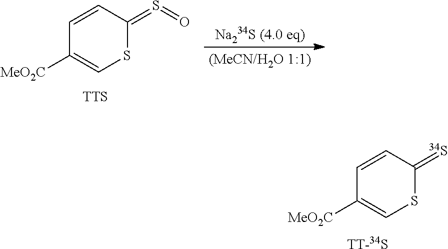

In a 20 mL vial, TTS (19.7 mg, 0.1 mmol, in 2 mL MeCN), and Na₂³⁴S solution (60 mM in H₂O, 2 mL) were added. The reaction was incubated at room temperature for 30 min, which was monitored by UV-Vis spectroscopy. The reaction was quenched by H₂O and extracted by EA. Combined organic layers were washed by brine and dried by Na₂SO₄. The solvent was removed by rotary evaporator. The residue was purified by flash column chromatography (HEX/EA=5/1) to yield TT-³⁴S (9.5 mg, yield=52%) as a dark red solid.

TT-³⁴S HRMS (ESI, m/z): [M+H]⁺ calculated for C₇H₇O₂S₃⁴S⁺: 188.9840. found: 188.9866.

Isotope labelling experiments as described herein were carried out using the following reaction.

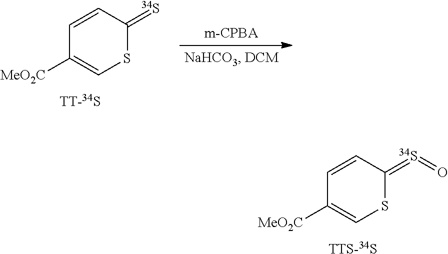

To a stirred solution of TT-³⁴S (6.2 mg, 0.03 mmol) in DCM (1 mL), NaHCO₃ (13 mg, 0.15 mmol, 5.0 eq) and m-CPBA (70%, 7.4 mg, 0.03 mmol, 1.0 eq) were added. After being stirred at room temperature for 30 min, the reaction was quenched by Sat. NaHCO₃ solution and extracted by EA. Combined organic layers were washed by brine and dried by Na₂SO₄. The solvent was removed by rotary evaporator. The residue was purified by flash column chromatography (HEX/EA=5/1-3/1) to yield TTS-³⁴S (4.8 mg, yield=71%) as a dark red powder.

TTS-³⁴S HRMS (ESI, m/z): [M+H]⁺ calculated for C₇H₇O₃S₃⁴S⁺: 204.9798. found: 204.9802.

Isotope labelling experiments as described herein were carried out using the following reaction.

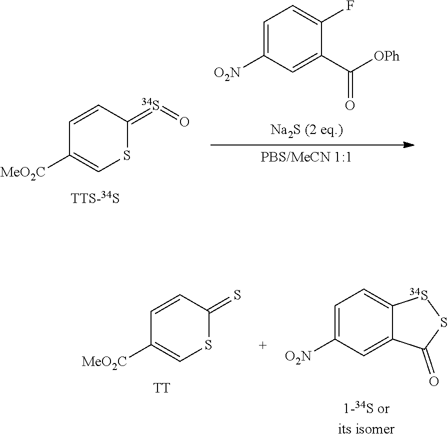

To a solution of TTS-³⁴S (4.1 mg, 0.02 mmol) in MeCN (2 mL), Na₂S solution (2.0 mL, 20 mM in PBS buffer) was added dropwise. After being incubated at room temperature for 10 min, phenyl 2-fluoro-5-nitrobenzoate (5.2 mg, 0.02 mmol) was added. The reaction was incubated for another 1 h. TT and 1-³⁴S were detected by LC-MS.

TT: HRMS (ESI, m/z): [M+H]⁺ calculated for C₇H₇O₂S₂⁺: 186.9882. found: 186.9891.

1-³⁴S: HRMS (ESI, m/z): [M+H]⁺ calculated for C₇H₄NO₃³⁴SS⁺: 215.9585. found: 215.9601.

### Example IX

### UV-Vis Detection of Reaction of TTS with H₂S

UV-Vis experiments as described herein were carried out using the following protocol.

TTS (20 μL, 10 mM in DMSO solution, final concentration=100 μM) and Na₂S (40 μL, 10 mM in H₂O, final concentration=200 μM), reacted in PBS buffer, pH=7.4 (2 mL) at room temperature. The data was collected by UV-Vis spectrometer (700-250 nm, every 1 min). There was a significant change of the maximum absorbance wavelength (from 380 nm to 332 nm).

### Example X

### Reaction Kinetics of TT Generation from the Reaction of TTS with H₂S

Kinetic study of the reaction between TTS and H₂S: TTS (10 μL, 10 mM in DMSO solution, final concentration=50 μM) and Na₂S (20 μL, 500 mM in MilliQ H₂O, final concentration=5 mM), reacted in PBS buffer (50 mM, pH=7.4) (2 mL) at room temperature. The kinetics were detected by UV-Vis absorbance spectrum (λ=380 nm) based on the decrease of TTS. The representative kinetic results are shown in FIG. 1BC. This reaction can be seen as a pseudo-first order reaction; the second-order k constant (k) was calculated by:

$${k}_{obs} = k [ {Na}_{2} S ] , where [ {Na}_{2} S ] = 0.0 \; 05 M$$

$${k}_{obs} t = - ln \frac{[ TTS ]}{{[ TTS ]}_{0}} , {where [ TTS ]}_{0} = 50 μM$$

$$k = 2.76 {M}^{- 1} {s}^{- 1}$$

The final k reported above was calculated by taking the average of the k values from 3 trials.
To determine [TTS] at each time point:

$$A = {ε}_{TTS} \times [ TTS ] + {e}_{TT} \times [ TT ]$$

$$Considering [ TTS ] + [ TT ] = 50 μM ,$$

$$A = {ε}_{TTS} \times [ TTS ] + {e}_{TT} \times ( 50 - [ TTS ] )$$

$${ε}_{TTS ( 380 nm )} = 0.0 \; 147 {μM}^{- 1} {cm}^{- 1} and {ε}_{TT ( 380 nm )} = 0.0 \; 025 {μM}^{- 1} {cm}^{- 1}$$

$$\frac{A - 0.125}{0.0122} = [ TTS ]$$

### Example XI

### SSP4 Fluorescence Assay to Determine Generation of H₂S₂

SSP4 was dissolved in DMSO to obtain a 200 μM stock solution. CTAB was dissolved in ethanol to yield a 2 mM stock solution. TTS and TT were dissolved in DMSO to make 1 mM stock solutions. Na₂S, Na₂S₂, and L-cysteine were prepared in MilliQ H₂O to obtain stock solutions at 2 mM, 1 mM, and 4 mM, respectively. 50 mM PBS buffer was added to individual wells of a black polystyrene, flat-bottomed clear 96-well plate, followed by TTS (25 μM) or TT (25 μM), and Na₂S (50 μM), cysteine (100 μM), or Na₂S₂ (25 μM). For the interference assay, TTS or TT (25 μM) were added to wells with or without Na₂S (50 μM). Samples were mixed by pipetting up and down and incubated in the dark at room temperature (rt) for 30 min. Next, CTAB (50 μM) was added to all wells followed by SSP4 (5 μM), and samples were mixed by pipetting up and down. The final volume was 200 μL in each well. Samples were then incubated in the dark at room temperature for another 30 min before being measured on a Molecular Devices SpectraMax iD3 Multi-Mode Microplate Reader (emission 525 nm; excitation 485 nm; integration time 400s, low PMT sensitivity, attenuation 1, read height 1.00 mm).

Monitoring H₂S₂ formation from the TTS-TAGDD hybrid compound 3: TTS and 3 were prepared as 1 mM solutions in DMSO. L-cysteine and Na₂S₂ were prepared at 4 mM and 1 mM in MilliQ H₂O, respectively. SSP4 was dissolved in DMSO to obtain a 200 μM stock solution. CTAB was dissolved in ethanol to yield a 2 mM stock solution. 50 mM PBS buffer (pH 7.4) was added to individual wells of a black polystyrene, flat-bottomed clear 96-well plate, followed by TTS (25 μM) or 3 (25 μM) then cysteine (100 μM) or Na₂S₂ (25 μM). Samples were mixed by pipetting up and down and incubated in the dark at room temperature (rt) for 30 min. Next, CTAB (50 μM) was added to all wells followed by SSP4 (5 μM), and samples were mixed by pipetting up and down. Samples were then incubated in the dark at room temperature for another 30 min before being measured on a Molecular Devices SpectraMax iD3 Multi-Mode Microplate Reader (fluorescence mode; kinetics measurement type; emission 535 nm; excitation 485 nm; integration time 400s, low PMT sensitivity, attenuation 1, read height 1.00 mm; total run time: 12 hours; 25 cycles; interval: 0.5 hours). Data was graphed as a time dependent fluorescence response, with F₀ as the SSP4 only condition. The fluorescence of Na₂S₂ over time was not represented in the line graph presented due to the compound's known instability. Data obtained from the 12 h time point including from Na₂S₂ was also represented in a bar graph for greater clarity. Results were expressed as mean±SEM (n=3).

### Example XII

### Cell Viability and Cytotoxicity Assay

HeLa cells were cultured in DMEM/F12 (1:1) (Gibco, Invitrogen, #11330-032) medium supplemented with 10% fetal bovine serum (FBS) at 37° C., 5% CO₂ overnight. Cells were then seeded at a density of 11,000 cells/well in the inner wells of a 96-well black/clear flat-bottomed plate (ThermoFisher Scientific, #165305) at 37° C., 5% CO₂ overnight. Media was then aspirated from wells, and cells were washed twice with 1×PBS. TTS and TT were first prepared at 10, 25, 50, and 100 mM concentrations in DMSO and then diluted in 10% FBS media to 10, 25, 50, and 100 μM (0.1% DMSO). Compounds were then added to cells (100 μL/well) and incubated for 24 hrs at 37° C., 5% CO₂. Afterwards, WST-8 from the CCK-8 assay was added to cells (10 μL) following the manufacturer's instructions and incubated for 2 hrs at 37° C., 5% CO₂. The absorbance at 450 nm was measured with the Molecular Devices SpectraMax iD3 Multi-Mode Microplate Reader. The optical density (OD) of the wells (3 wells per condition) was used to calculate the relative cell viability (%) according to the following formula:

$$Cell Viability ( \% ) = ( ODtreatment group - {OD}_{blank} / ODcontrol group - {OD}_{blank} ) * 100$$

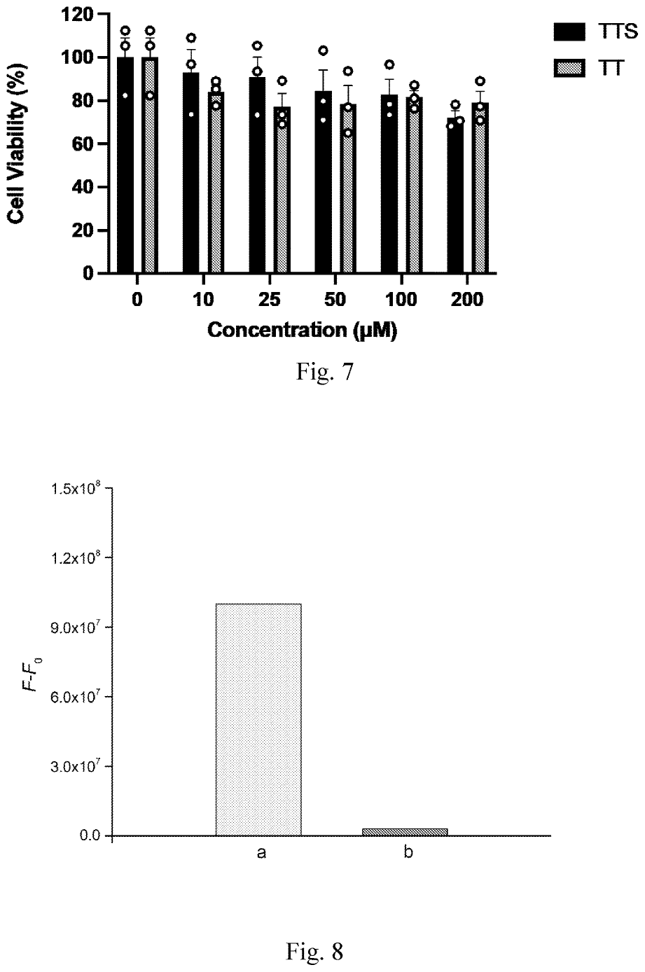

Cell viability of TTS and TT in HeLa cells with the CCK-8 assay is shown in FIG. 7 Results are expressed as mean±SEM (n=3).

### Example XIII

### Protein-Sulfine Compound Conjugates and Protein Labelling of TTS

Preparation of lysozyme-TTS conjugate: To 600 μL solution of chicken lysozyme (14.3 kDa, 300 μM in 1×PBS buffer, pH 9), 2 (30 μL, 60 mM in DMSO, 10 eq) was added. The mixture was incubated at room temperature for 16 h to produce Lyso-TTS as shown in FIG. 8. Excess 2 was removed by a Zeba 7K MWCO (ThermoFisher Scientific) spin column using PBS buffer pH 7.4 solution as the eluent. The final concentration of Lyso-TTS was 80 μM as determined by Nanodrop Spectrophotometer. Lyso-TTS was characterized by LC-MS ([M+H]⁺ 14607.11)

The reaction between lysozyme-TTS and H₂S: To a solution of Lyso-TTS (80 μM, 300 μL in PBS buffer pH 7.4), Na₂S (30 μL, 10 mM in PBS pH 7.4 buffer) was added. The mixture was incubated at room temperature for 30 min to give Lyso-TT as shown in FIG. 5 which was then purified by a Zeba 7K MWCO spin column using PBS buffer pH 7.4 as the eluent. The final concentration of the modified lysozyme (Lyso-TT) was 10 μM as determined by Nanodrop Spectrophotometer. Lyso-TT was characterized by LC-MS ([M+H]⁺: 14591.33).

Fluorescence analysis of H₂S₂ generated from the reaction between Lyso-TTS and H₂S: In the Lyso-TTS/Na₂S experiment described above, 50 μL of the reaction solution (after Lyso-TTS and Na₂S were mixed and incubated for 30 min) was taken and treated with SSP4 to demonstrate the formation of H₂S₂ in this process (see FIG. 8). The results showed 30-fold florescence increase in group a compared to group b, which means the significance H₂S₂ release in the reaction between Lyso-TTS and H₂S.

### Example XIV

### GAPDH Protein S-Persulfidation

216 μL of PBS (50 mM, pH 7.4) was added to 3.9 mg of GAPDH (from rabbit muscle, #G2267, Sigma-Aldrich) to obtain a concentration of 500 μM (MW: 36 kDa). DTT (10 mM in DMSO, 11.35 μL) was added for a final equal concentration with the GAPDH. The solution was incubated in the dark at room temperature for 30 min. Zeba 7K MWCO spin columns were prepared and used according to manufacturer's instructions with 3 PBS washes. Excess reductant was removed by column purification with the Zeba 7K MWCO columns. The resulting reduced protein concentration was determined by the absorbance at 280 nm (εmM=30.4 mM-1 cm-1) using the Nanodrop One Microvolume UV-Vis Spectrophotometer (ThermoFisher Scientific). For controls, reduced GAPDH was treated with NEM (1:5 equiv.) and incubated at rt in the dark for 30 min before excess NEM was removed by the Zeba 7K MWCO columns. Next, the reduced protein was diluted to a final concentration of 100 μM in a final volume of 100 μL and treated with TTS (10 μL of 10 mM in DMSO for a final concentration of 1 mM), Na₂S (10 μL of 10 mM in MilliQ H₂O for a final concentration of 1 mM), both TTS and Na₂S, or Na₂S₂ (10 μL of 10 mM in MilliQ H₂O for a final concentration of 1 mM). For example, if the reduced protein (361 μM) was only to be treated with TTS, 62.3 μL of PBS (50 mM, pH 7.4) would first be added to an Eppendorf tube, followed by 27.7 μL of protein, and followed by 10 μL of TTS. TTS+Na₂S and Na₂S₂ samples were also prepared at the concentrations above without any protein, where PBS (50 mM, pH 7.4) was used in place of the proteins (volume-wise). Samples were incubated at room temperature for 30 min in the dark before being purified by Zeba 7K MWCO columns (three times to ensure proper removal of excess treatment molecules) and quantified by absorbance. For samples that did not contain protein (and couldn't be quantified by Nanodrop), volumes used followed those of their respective samples containing protein. All samples were then incubated with SSP4 (5 μL of 100 μM in DMSO for a final concentration of 5 μM) in individual wells of a black clear flat bottom 96-well plate. The final volume was 100 μL. For example, if the persulfidated protein (99.5 μM) was to be examined, 84.95 μL of PBS (50 mM, pH 7.4) would first be added to a well, followed by 10.05 μL of protein, and then followed by 5 μL of SSP4. Incubation occurred at room temperature in the dark for 30 min before being measured on the SpectraMax iD3 microplate reader (ex: 485 nm; em: 525 nm).

### Example XV

### TTS Reaction with Different H₂S Donors

The present disclosure contemplates use of TTS and an H₂S donor molecule (which generates H₂S) to generate H₂S₂. TTS was tested with H₂S donor compound JK-1, which is a hydrolysis-based H₂S donor which has shown promising activities in cardiovascular disease models.

Measurement of H₂S₂ formation from JK-1 with TTS: JK-1 (1.1 mg) was added to a 1 dram vial and 4.484 mL of 50 mM PBS (pH 6.0) was then added to obtain a 1 mM stock solution with no headspace in the vial. The solution was vortexed to mix and then incubated in the dark at rt for 20 min. Based on the H₂S-release profile reported under the same conditions (see Kang, J. et al., J. Am. Chem. Soc. 2016, 138, 6336), 400 μM of H₂S (40% of 1 mM) should be released. The pH of the solution was then adjusted to 7.4 by adding 16 μL 2N NaOH. The same volume of 50 mM PBS (pH 6.0) buffer was added to an empty 1 dram vial and 16 μL of 2N NaOH was added to adjust the pH of the buffer similarly for the controls. After incubation, 500 μL of the pH-adjusted PBS was aliquoted to nine 1.5 mL Eppendorf tubes while the same volume of JK-1 solution was aliquoted into six 1.5 mL Eppendorf tubes. TTS (10 mM stock in DMSO, 10 μL) was then added to three of the JK-1-containing tubes and to three of the pH-adjusted PBS only tubes. Na₂S₂ (10 mM stock in MilliQ H₂O, 10 μL) was added to three of the pH-adjusted PBS only tubes. Tubes were closed and vortexed to mix. Incubation occurred for 30 min at room temperature (“rt”) in the dark. CTAB (100 mM stock in EtOH, 2 μL) was then added to all 15 tubes followed by SSP4 (9.44 mM stock in DMSO, 2.11 μL). Tubes were closed, vortexed to mix, and incubated for 30 min in the dark at rt. 200 μL of each tube was then added to separate wells on a 96-well black flat clear-bottomed plate before being measured on a Molecular Devices SpectraMax iD3 Multi-Mode Microplate Reader (emission 535 nm; excitation 485 nm; integration time 400s, low PMT sensitivity, attenuation 1, read height 3.00 mm).

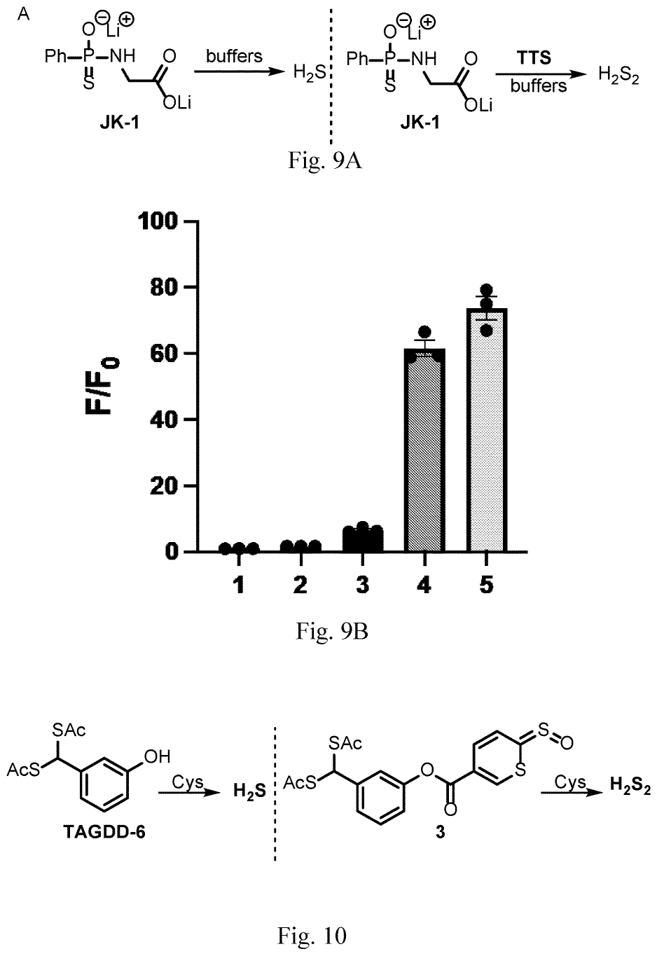

As shown in FIG. 9A, JK-1 when placed in a buffer generates H₂S. JK-1 in buffer was mixed with TTS to generate H₂S₂. After a 30 min incubation, the formation of H₂S₂ was measured by SSP4. As shown in FIG. 9B, only the co-existence of JK-1 and TTS gave strong fluorescence, which was comparable to the signal obtained from Na₂S₂.

### Example XVI

### H₂S Donor—TTS Conjugates for Generating H₂S₂

The present disclosure contemplates a conjugate of TTS and an H₂S donor for generation of H₂S₂. A TTS-TAGDD hybrid compound 3 (see FIG. 10) was synthesized where the H₂S donor was covalently attached to TTS. TAGDD is a gem-dithiol based H₂S donor which can be triggered by biothiols such as cysteine to release H₂S. See Zhao, Y. et al., Thio-activated gem-dithiols: A new class of controllable hydrogen sulfide donors. Org. Lett. 2014, 16, 4536-4539. According to the present disclosure, cysteine is used to trigger the release of H₂S from the TAGDD component of 3. The H₂S in close proximity to TTS, reacts with TTS to generate H₂S₂ as described herein.

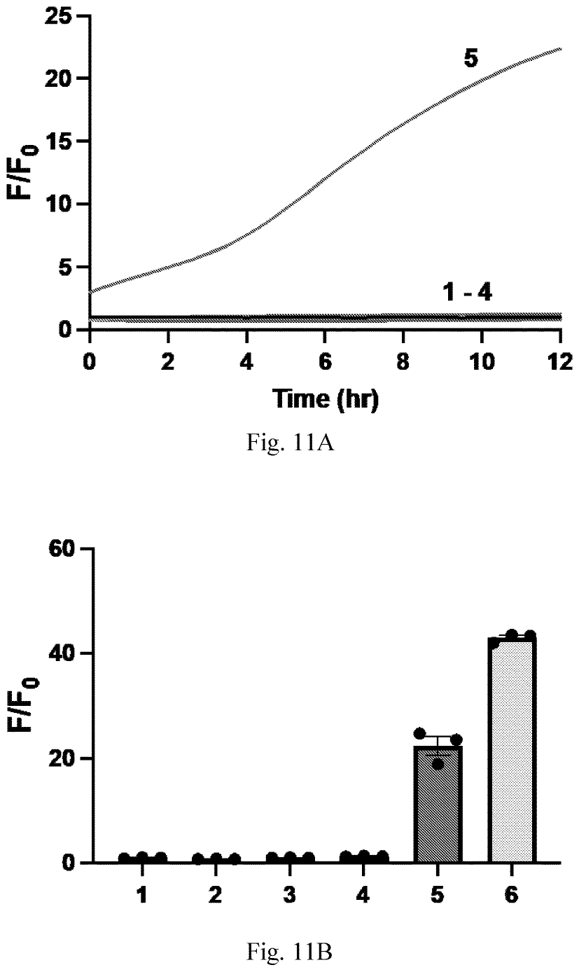

As shown in FIG. 11A and FIG. 11B, when the conjugate compound 3 was treated with Cys, steady and continuous H₂S₂ formation (monitored by SSP4 signals) was observed. Control experiments (e.g. conjugate compound 3 only or TTS only) did not give any notable fluorescence. Results shown in FIGS. 11A and 11B clearly demonstrate that TTS can boost H₂S₂ formation from H₂S donors.

### Example XVII

### Synthesis and Characterization of a Sulfine-Based Fused Ring Structure TTS-2

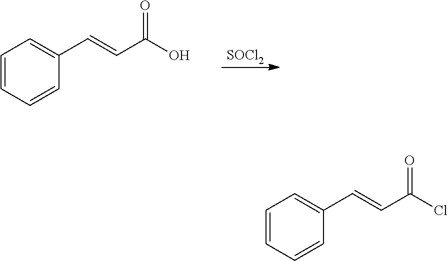

To an oven-dried round bottom flask with a magnetic stir bar was added cinnamic acid (2.00 g, 13.5. mmol) and thionyl chloride (15 mL, 207 mmol). The flask was sealed with a rubber septum, evacuated and backfilled with argon. After being stirred at room temperature for 24 h, the reaction mixture was concentrated in vacuo. To the obtained residue was added toluene (30 mL), and the mixture was concentrated in vacuo to yield cinnamoyl chloride as a yellow solid. Note: This product was directly used in the next step without further purification.

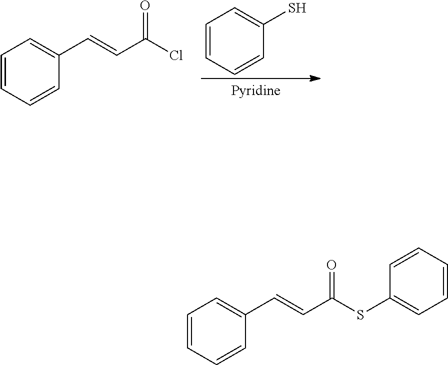

To an oven-dried round bottom flask with a magnetic stir bar was added cinnamoyl chloride (2.00 g, 12.0 mmol) and pyridine (20 mL). The flask was sealed with a rubber septum, evacuated and backfilled with argon. The reaction mixture was cooled to 0° C., and benzenethiol (1.32 g, 12.0 mmol) was added slowly via syringe. After being stirred at room temperature for 24 h, the reaction mixture was quenched with water. The aqueous solution was extracted with EtOAc. The combined organic layer was washed with brine, dried over Na₂SO₄, filtered and concentrated in vacuo. The resulting residue was purified via flash chromatography (0-5% EtOAc/Hexanes) to yield S-phenyl-3-phenylprop-2-enethioate (2.00 g, yield=69.4%) as a white solid.

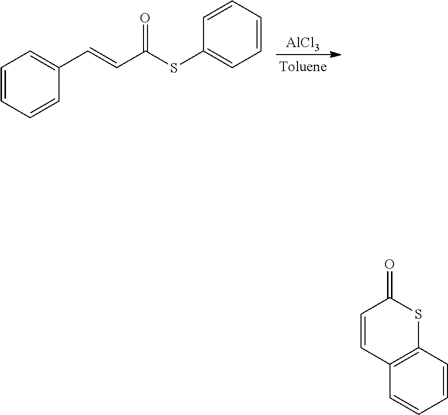

To an oven-dried round bottom flask with a magnetic stir bar was added S-phenyl-3-phenylprop-2-enethioate (4.00 g, 16.7 mmol), aluminum trichloride (5.54 g, 41.6 mmol), and toluene (100 mL). The flask was sealed with a rubber septum, evacuated and backfilled with argon. The reaction mixture was heated to 100° C. for 24 h. After cooling to room temperature, the reaction mixture was quenched with ice water. The aqueous solution was extracted with EtOAc. The combined organic layer was washed with brine, dried over Na₂SO₄, filtered and concentrated in vacuo. The resulting residue was purified via flash chromatography (0-5% EtOAc/Hexanes) to yield 2H-thiochromen-2-one (880 mg, yield=32.5%) as a white solid.

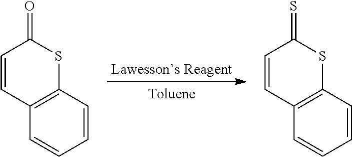 [SMILES: O=c1ccc2ccccc2s1 ; S=c1ccc2ccccc2s1]

To an oven-dried round bottom flask with a magnetic stir bar was added 2H-thiochromen-2-one (500 mg, 3.08 mmol), Lawesson's Reagent (1.25 g, 3.08 mmol), and toluene (50 mL). The flask was sealed with a rubber septum, evacuated and backfilled with argon. The reaction mixture was heated to 100° C. for 72 h. After cooling to room temperature, the reaction mixture was concentrated in vacuo. The obtained residue was purified via flash chromatography (0-20% EtOAc/Hexanes) to yield 2H-thiochromen-2-thione (468 mg, yield=85.3%) as a red solid.

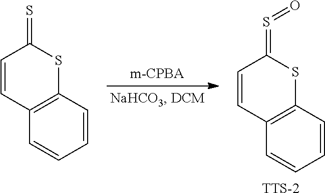

To an oven-dried round bottom flask with a magnetic stir bar was added 2H-thiochromen-2-thione (200 mg, 1.12 mmol), m-CPBA (70%, 248 mg, 1.01 mmol), NaHCO₃ (470 mg, 5.60 mmol), and DCM (30 mL). After being stirred at room temperature for 30 min, an aqueous solution of saturated NaHCO₃ was added. The aqueous solution was extracted with DCM. The combined organic layer was washed with brine, dried over Na₂SO₄, filtered and concentrated in vacuo. The resulting residue was purified via flash chromatography (0-30% EtOAc/Hexanes) to yield (2H-thiochromen-2-ylidene)-λ⁴-sulfanone (172 mg, yield=87.6%) as a dark red solid.

### Example XVIII

### Synthesis and Characterization of Sulfine-Based Five-Membered Ring Structures TTS-3 and TTS-6

### Synthesis of TTS-3

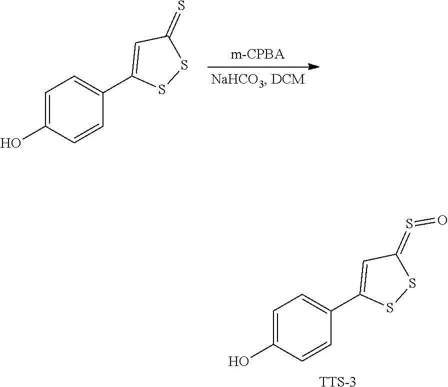

To an oven-dried round bottom flask with a magnetic stir bar was added 5-(4-hydroxyphenyl)-3H-1,2-dithiole-3-thione (200 mg, 0.88 mmol), m-CPBA (70%, 195 mg, 0.79 mmol), NaHCO₃ (370 mg, 4.40 mmol), and DCM (100 mL). After being stirred at room temperature for 30 min, an aqueous solution of saturated NaHCO₃ was added. The reaction mixture was filtered and the precipitate was collected and washed with deionized water to yield (5-(4-hydroxyphenyl)-3H-1,2-dithiol-3-ylidene)-λ⁴-sulfanone (60 mg, yield=31.4%) as a dark red solid.

TTS-3: ¹H NMR (600 MHz, DMSO-d₆) δ 10.34 (s, 1H), 7.87 (s, 1H), 7.59 (d, J=8.2 Hz, 2H), 6.87 (d, J=8.2 Hz, 2H). ¹³C NMR (151 MHz, DMSO-d₆) δ 202.41, 160.46, 160.40, 128.91, 122.85, 116.28, 114.24. HRMS (ESI, m/z): [M]⁺ calculated for C₉H₆O₂S₃⁺: 241.9530. found: 241.9516.

### Synthesis of TTS-6

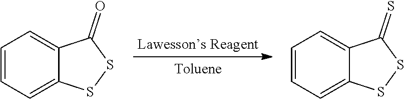 [SMILES: O=c1ssc2ccccc12 ; S=c1ssc2ccccc12]

To an oven-dried round bottom flask with a magnetic stir bar was added 3H-benzo[c][1,2]dithiol-3-one (1.00 g, 5.95 mmol), Lawesson's Reagent (1.60 g, 3.96 mmol), and toluene (100 mL). The flask was sealed with a rubber septum, evacuated and backfilled with argon. The reaction mixture was heated to 100° C. for 72 h. After cooling to room temperature, the reaction mixture was concentrated in vacuo. The obtained residue was purified via flash chromatography (0-10% EtOAc/Hexanes) to yield 3H-benzo[c][1,2]dithiole-3-thione (875 mg, yield=79.9%) as an orange solid.

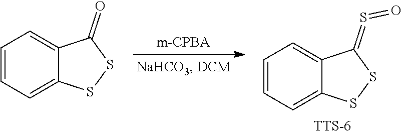

To an oven-dried round bottom flask with a magnetic stir bar was added 3H-benzo[c][1,2]dithiole-3-thione (200 mg, 1.09 mmol), m-CPBA (70%, 240 mg, 0.98 mmol), NaHCO₃ (458 mg, 5.45 mmol), and DCM (30 mL). After being stirred at room temperature for 30 min, an aqueous solution of saturated NaHCO₃ was added. The aqueous solution was extracted with DCM. The combined organic layer was washed with brine, dried over Na₂SO₄, filtered and concentrated in vacuo. The resulting residue was purified via flash chromatography (0-25% EtOAc/Hexanes) to yield (3H-benzo[c][1,2]dithiol-3-ylidene)-λ⁴-sulfanone (184 mg, yield=93.9%) as a red solid.

### Example XIX

### General and Computational Methods

All the reactions were stirred using Teflon-coated magnetic stir bars. Elevated temperatures were maintained using Thermostat-controlled silicone oil baths. Chemicals were purchased from Aldrich, Alfa Aesar, TCI, and used without further purification, unless otherwise indicated. Analytical TLC was performed with 0.25 mm silica gel G plates with a 254 nm fluorescent indicator. The TLC plates were visualized by ultraviolet light and treatment with anisaldehyde-H₂SO₄ stain followed by gentle heating. Purification of products was accomplished by flash chromatography on silica gel and the purified compounds show a single spot by analytical TLC.

NMR spectra were measured on Bruker high field NMR spectrometers (¹H at 400 MHz or 600 MHz, ¹³C at 101 MHz or 151 MHz) nuclear magnetic resonance spectrometers. Data for ¹H-NMR spectra are reported as follows: chemical shift (ppm, referenced to residual solvent peak CDCl₃: 7.26 ppm, CD₂Cl₂: 5.32 ppm, CD₃OD: 3.31 ppm); s=singlet, d=doublet, t=triplet, q=quartet, dd=doublet of doublets, m=multiplet), coupling constant (Hz), and integration. Data for ¹³C-NMR are reported in terms of chemical shift (ppm) relative to residual solvent peak (CDCl₃: 77.16 ppm, CD₂Cl₂: 53.84 ppm, CD₃OD: 49.00 ppm). High-resolution mass spectra (HRMS) were recorded on Agilent 6530 LC-MS (ESI). Absorption spectra were recorded on a Thermo Scientific Evolution 300 UV-Vis Spectrophotometer using 1 cm quartz cells. Fluorescence excitation and emission spectra were measured on a Cary Eclipse fluorescence spectrophotometer. Cell imaging was performed using the Keyence All-in-One Fluorescence Microscope (BZ-X810).

All calculations were performed with the Gaussian 09, Revision D.01 program. Geometry optimizations of all minima and transition states involved were carried out using M06-2X⁵ (see Zhao, Y. et al., Theor. Chem. Acc. 2008, 120, 215), functional and SMD⁶ (see Marenich, A. V. et al., J. Phys. Chem. B, 2009, 113, 6378) solvation model in H₂O solvent and the basis set was maug-cc-pVTZ (see Papajak, E. et al., Efficient Diffuse Basis Sets: cc-pVxZ+ and maug-cc-pVxZ. J. Chem. Theory Comput. 2009, 5, 3330). It was labelled as SMD(H₂O)/M06-2X/maug-cc-pVTZ level. Frequency calculations at the same level were performed to validate each structure as either a minimum (the number of imaginary frequencies=0) or a transition state (the number of imaginary frequencies=1) and to evaluate its zero-point energy and thermal corrections at 298 K. Standard states are the hypothetical states at 1 mol/L.

It should be emphasized that the above-described embodiments and specific examples of the present invention, particularly, any “preferred” embodiments, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

1. A method of generating H₂S₂ comprising
reacting a compound comprising a sulfine moiety with H₂S directly or generated by an H₂S donor to generate H₂S₂.

2. The method of claim 1 wherein the compound comprising a sulfine moiety further comprises an aromatic heterocycle.

3. The method of claim 1 wherein the compound comprising a sulfine moiety further comprises an aromatic heterocycle substituted at one or more locations on the heterocycle.

4. The method of claim 1 wherein the compound comprising a sulfine moiety further comprises an aromatic heterocycle substituted at one or more locations on the heterocycle with a carboxyl group or an ester.

5. The method of claim 1 wherein the compound comprising a sulfine moiety further comprises an aromatic six-membered heterocycle wherein the carbon atom of the sulfine moiety is within the heterocycle and the heterocycle substituted at the C4 location with a carboxyl group or an ester.

6. The method of claim 1 wherein the compound comprising a sulfine moiety is compound 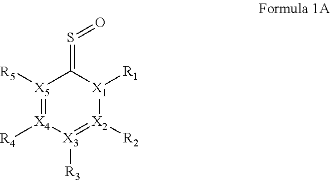
X₁ is an oxygen atom, a nitrogen atom, a sulfur atom or a carbon atom, wherein R₁ is absent when X₁ is oxygen, sulfur or double bonded nitrogen;
X₂ is an oxygen atom, a nitrogen atom, a sulfur atom or a carbon atom, wherein R₂ is absent when X₂ is oxygen, sulfur or double bonded nitrogen;
X₃ is an oxygen atom, a nitrogen atom, a sulfur atom or a carbon atom, wherein R₃ is absent when X₃ is oxygen, sulfur or double bonded nitrogen;
X₄ is an oxygen atom, a nitrogen atom, a sulfur atom or a carbon, atom wherein R₄ is absent when X₄ is oxygen, sulfur or double bonded nitrogen;
X₅ is an oxygen atom, a nitrogen atom, a sulfur atom or a carbon atom, wherein R₅ is absent when X₅ is oxygen, sulfur or double bonded nitrogen;
with the proviso that no more than two of X₁, X₂, X₃, X₄, X₅ may be an atom other than carbon, i.e. a heteroatom,
R₁ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker;
R₂ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker;
R₃ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker;
R₄ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker; and
R₅ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker.
R₁, R₂, R₃, R₄, and R₅ may be the same or different,
wherein R₁, R₂, R₃, R₄, or R₅ or the linker may optionally be bound to a protein, antibody, receptor, ligand, antibody, delivery molecule, device, particle or H₂S donating compound.

7. The method of claim 6 wherein the compound is

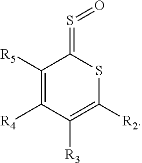

8. The method of claim 6 wherein the compound is 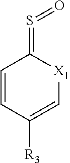
where X₁ is sulfur, oxygen or nitrogen.

9. The method of claim 6 wherein the compound is

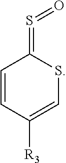

10. The method of claim 6 wherein the compound is

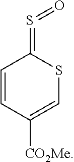 [SMILES: COC(=O)c1ccc(=S=O)sc1]

11. The method of claim 6 wherein the compound is

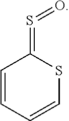

12. The method of claim 1 wherein the compound comprising a sulfine moiety is attached to a delivery agent for a location-specific site within a tissue or cell for generating H₂S₂ at the location-specific site.

13. The method of claim 1 wherein the compound comprising a sulfine moiety is attached to a protein.

14. The method of claim 1 wherein the compound comprising a sulfine moiety is attached to an H₂S donor.

15. The method of claim 1 wherein the compound comprising a sulfine moiety reacts with H₂S and oxygen of the sulfine moiety is removed producing a compound comprising a C═S moiety.

16. The method of claim 1 wherein the compound comprising a sulfine moiety reacts with H₂S and oxygen of the sulfine moiety is removed producing a compound comprising a C═S moiety and wherein the compound comprising a C═S moiety reacts with an oxidant to regenerate the compound with a sulfine moiety.

17. The method of claim 1 wherein the compound comprising a sulfine moiety is introduced to a biological site within an individual.

18. The method of claim 1 wherein the compound comprising a sulfine moiety is introduced to cells within an individual.

19. A compound of formula 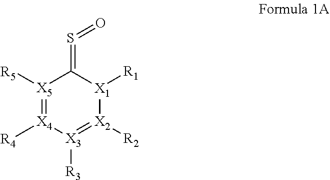
X₁ is an oxygen atom, a nitrogen atom, a sulfur atom or a carbon atom, wherein R₁ is absent when X₁ is oxygen, sulfur or double bonded nitrogen;
X₂ is an oxygen atom, a nitrogen atom, a sulfur atom or a carbon atom, wherein R₂ is absent when X₂ is oxygen, sulfur or double bonded nitrogen;
X₃ is an oxygen atom, a nitrogen atom, a sulfur atom or a carbon atom, wherein R₃ is absent when X₃ is oxygen, sulfur or double bonded nitrogen;
X₄ is an oxygen atom, a nitrogen atom, a sulfur atom or a carbon, atom wherein R₄ is absent when X₄ is oxygen, sulfur or double bonded nitrogen;
X₅ is an oxygen atom, a nitrogen atom, a sulfur atom or a carbon atom, wherein R₅ is absent when X₅ is oxygen, sulfur or double bonded nitrogen;
with the proviso that no more than two of X₁, X₂, X₃, X₄, X₅ may be an atom other than carbon, i.e. a heteroatom,
R₁ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker;
R₂ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker;
R₃ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker;
R₄ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker; and
R₅ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker.
R₁, R₂, R₃, R₄, and R₅ may be the same or different,
wherein R₁, R₂, R₃, R₄, or R₅ or the linker may optionally be bound to a protein, antibody, receptor, ligand, antibody, delivery molecule, device, particle or H₂S donating compound.

20. The compound of claim 19 being

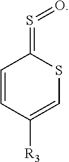

21. The compound of claim 19 being

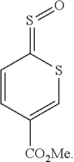 [SMILES: COC(=O)c1ccc(=S=O)sc1]

22. The compound of claim 19 being

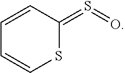

23. The compound of claim 19 being

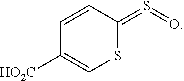 [SMILES: O=S=c1ccc(C(=O)O)cs1]

24. The compound of claim 19 being

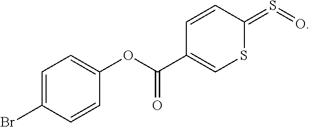 [SMILES: O=S=c1ccc(C(=O)Oc2ccc(Br)cc2)cs1]

25. The compound of claim 20 being

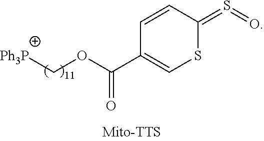

26. The compound of claim 19 wherein
R₅ and R₄ is part of a fused ring structure such as a C5-C7 cyclic structure, saturated or unsaturated, heterocyclic or nonheterocyclic, substituted or unsubstituted, or
R₄ and R₃ is part of a fused ring structure such as a C5-C7 cyclic structure, saturated or unsaturated, heterocyclic or nonheterocyclic, substituted or unsubstituted, or
R₃ and R₂ is part of a fused ring structure such as a C5-C7 cyclic structure, saturated or unsaturated, heterocyclic or nonheterocyclic, substituted or unsubstituted.

27. The compound of claim 26 being

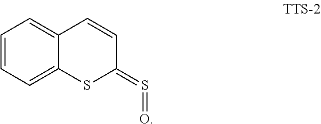 [SMILES: O=S=c1ccc2ccccc2s1]

28. A compound of formula 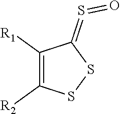
wherein
R₁ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker,
R₂ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker, wherein
R₁ and R₂ can optionally be part of a fused ring structure such as a C5-C7 cyclic structure, saturated or unsaturated, heterocyclic or nonheterocyclic, substituted or unsubstituted.

29. The compound of claim 28 being

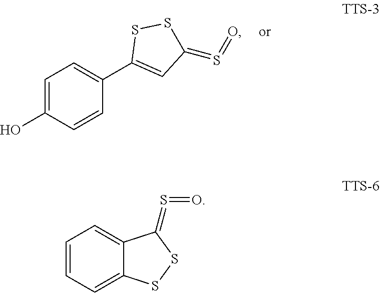 [SMILES: O=S=c1cc(-c2ccc(O)cc2)ss1 ; O=S=c1ssc2ccccc12]

30. The compound of claim 19 attached to a protein.

31. The compound of claim 19 attached to an H₂S donating compound.

32. A pharmaceutical composition comprising a compound of claim 19 and a pharmaceutically acceptable vehicle or excipient.

33. The pharmaceutical composition of claim 32 wherein the compound is attached to a protein or an H₂S donating compound.

34. A method of persulfidation of a target protein comprising
reacting a compound of claim 19 with H₂S to produce H₂S₂ in the presence of a protein, and
wherein the H₂S₂ persulfidates the target protein.

35. A method of treating a patient comprising administering a compound of claim 19 to the patient in a therapeutically effective amount to generate a therapeutically effective amount of H₂S₂.

36. The method of claim 1 wherein the compound is

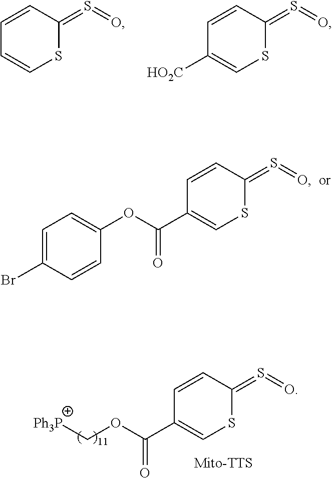

37. The method of claim 6 wherein
R₅ and R₄ is part of a fused ring structure such as a C5-C7 cyclic structure, saturated or unsaturated, heterocyclic or nonheterocyclic, substituted or unsubstituted, or
R₄ and R₃ is part of a fused ring structure such as a C5-C7 cyclic structure, saturated or unsaturated, heterocyclic or nonheterocyclic, substituted or unsubstituted, or
R₃ and R₂ is part of a fused ring structure such as a C5-C7 cyclic structure, saturated or unsaturated, heterocyclic or nonheterocyclic, substituted or unsubstituted.

38. The method of claim 37 wherein the compound of claim is

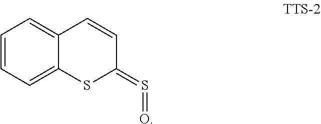 [SMILES: O=S=c1ccc2ccccc2s1]

39. The method of claim 1 wherein the compound is of formula 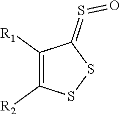
wherein
R₁ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker,
R₂ is hydrogen, carboxyl, ester, acyl, sulfonyl, phosphoryl, alkyl, substituted alkyl, halogen, aryl, substituted aryl, heteroaryl, alkenyl, substituted alkenyl, alkynyl, substituted alkynyl, cycloalkyl, heterocyclic, or a heteroatom or a linker, wherein
R₁ and R₂ can optionally be part of a fused ring structure such as a C5-C7 cyclic structure, saturated or unsaturated, heterocyclic or nonheterocyclic, substituted or unsubstituted.

40. The method of claim 39 wherein the compound is 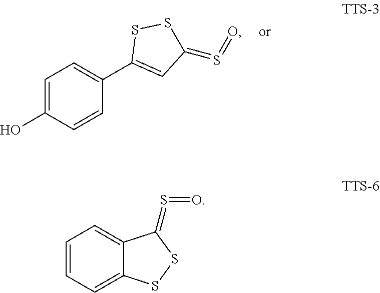 [SMILES: O=S=c1cc(-c2ccc(O)cc2)ss1 ; O=S=c1ssc2ccccc12]
